(19) United States
(12) Patent Application Publication
(10) Pub. No.: US 2021/0340617 A1
(43) Pub. Date: Nov. 4, 2021

(54) ENDOMETRIOSIS-ASSOCIATED GENETIC MARKERS PREDICT RESPONSIVENESS TO LEUPROLIDE ACETATE

(71) Applicant: JUNEAU BIOSCIENCES, L.L.C., Salt Lake City, UT (US)

(72) Inventors: Kenneth WARD, Salt Lake City, UT (US); VeeAnn ARGYLE, Salt Lake City, UT (US); Peeches CEDERHOLM, Salt Lake City, UT (US); Rakesh CHETTIER, Salt Lake City, UT (US)

(21) Appl. No.: 17/282,258

(22) PCT Filed: Oct. 4, 2019
(86) PCT No.: PCT/US19/54789

§ 371 Date: Apr. 1, 2021

Related U.S. Application Data
(60) Provisional application No. 62/741,432, filed on Oct. 4, 2018.

(51) Int. Cl. C12Q 1/6874; C12Q 1/6883; G16B 20/20

(52) U.S. Cl. CPC C12Q 1/6874; C12Q 1/6883; C12Q 2600/158; C12Q 2600/106; C12Q 2600/154; G16B 20/20

(57) ABSTRACT

Disclosed herein are methods of using genetic variants to select for an effective treatment of endometriosis, for example via a computer-implemented program to predict responsiveness of a subject to a selected treatment, and methods of diagnosing endometriosis or a symptom thereof.

### CROSS REFERENCE

This application claims the benefit of U.S. Provisional Application No. 62/741,432 filed Oct. 4, 2018, which is incorporated by reference herein in its entirety.

### BRIEF SUMMARY

An aspect of the disclosure provides a method that may comprise (a) detecting a presence of at least one genetic variant of Table 1 in a genetic material obtained from a subject, wherein the subject has endometriosis or is at risk of developing endometriosis; and (b) treating the subject for the endometriosis with a therapeutically effective amount of a treatment that comprises leuprolide acetate, a derivative thereof, a biosimilar thereof, or an interchangeable thereof.

An aspect of the disclosure provides a method that may comprise (a) detecting a presence of at least one genetic variant of Table 2 in a genetic material obtained from a subject, wherein the subject has endometriosis or is at risk of developing endometriosis; and (b) treating the subject for the endometriosis with a therapeutically effective amount of a treatment that does not comprise leuprolide acetate.

An aspect of the disclosure provides a method that may comprise detecting a presence of at least one genetic variant of Table 1 in a genetic material obtained from a subject, wherein the subject has endometriosis or is at risk of developing endometriosis, wherein the presence of the at least one genetic variant of Table 1 is indicative of a therapeutically effective response to a treatment for treating the endometriosis, and wherein the treatment comprises leuprolide acetate, a derivative thereof, a biosimilar thereof, or an interchangeable thereof.

An aspect of the disclosure provides a method that may comprise detecting a presence of at least one genetic variant of Table 2 in a genetic material obtained from a subject, wherein the subject has endometriosis or is a risk of developing endometriosis, wherein the presence of the at least one genetic variant of Table 2 is indicative of a therapeutically effective response to a treatment for treating the endometriosis, wherein the treatment does not comprise leuprolide acetate.

In some cases, the method may further comprise treating the subject for the endometriosis. In some cases, the treating may comprise prophylactic treating. In some cases, the method may further comprise altering or updating the treatment based at least in part on the detecting. In some cases, the detecting may occur prior to administering the treatment to the subject. In some cases, the method may further comprise selecting the treatment from a plurality of treatments. In some cases, the method may further comprise obtaining the genetic material from the subject. In some cases, the method may further comprise providing a recommendation to prescribe the treatment to the subject.

In some cases, the subject may have the endometriosis. In some cases, the subject may be at risk of developing the endometriosis. In some cases, the subject may suffer from pelvic pain. In some cases, the subject may suffer from infertility.

In some cases, the genetic material may be obtained from a reproductive tissue, a blood sample, or a combination thereof. In some cases, the genetic material may be obtained from the reproductive tissue that comprises endometrial tissue, uterine tissue, ovarian tissue, fallopian tissue, cervical tissue, vulvar tissue, or any combination thereof. In some cases, the genetic material may be obtained from the reproductive tissue that comprises the endometrial tissue. In some cases, the genetical material may be obtained from the blood sample. In some cases, the genetic material may comprise cell-free DNA. In some cases, the genetic material may comprise RNA.

In some cases, the genetic variant may comprise at least two genetic variants. In some cases, the genetic variant may be of MAP3K15. In some cases, the genetic variant may be of C17orf53, MTL5, SYT15, BCO2, ADD1, C14orf79, or any combination thereof.

In some cases, the detecting may comprise sequencing at least a portion of the genetic material. In some cases, the detecting may comprise hybridizing a probe to a portion of the genetic material, wherein the probe is specific for the genetic variant. In some cases, the method may further comprise measuring a total variant burden in at least a portion of the genetic material. In some cases, the method may further comprise measuring a mood of the subject. In some cases, the method may further comprise measuring a hormone receptor level in the genetic material. In some cases, the hormone receptor level may be an estrogen receptor level, a progesterone receptor level, or a combination thereof. In some cases, the hormone receptor level may be the estrogen receptor level. In some cases, the hormone receptor level may be the progesterone receptor level.

In some cases, the treatment may comprise administration of a gonadotropin releasing hormone (GnRH) or a synthetic analog thereof to the subject. In some cases, the treatment may comprise administration of a GnRH receptor agonist, a GnRH receptor antagonist, a progestin, norethindrone, medroxyprogesterone, a biosimilar of any of these, an interchangeable of any of these, a salt of any of these, or any combination thereof. In some cases, the treatment may comprise administration of RU-486 (CAS #84371-65-3), ethylnorgestrienone (CAS #16320-04-0), 2,3-isoxazolethisterone (CAS #17230-88-5), elagolix (CAS #834153-87-6), goserelin (CAS #65807-02-5), norethindrone acetate (CAS #38673-38-0), methylhydroxyprogesterone acetate (CAS #71-58-9), a biosimilar of any of these, an interchangeable of any of these, a salt of any of these, or any combination thereof. In some cases, the treatment may comprise administration of a pharmaceutical composition in unit dose form. In some cases, the treatment may comprise administration of a stem cell. In some cases, the treatment may comprise administration of composition comprising: a cannabis, a nonsteroidal anti-inflammatory drug (NSAID), a progestin, a progesterone, or any combination thereof. In some cases, the composition may comprise the cannabis, the NSAID, and the progestin. In some cases, the composition may comprise the cannabis, the NSAID, and the progesterone. In some cases, the NSAID may comprise ibuprofen, naproxen, or a combination thereof. In some cases, the composition may further comprise human serum albumin.

In some cases, the method may further comprise comparing a result of the method to a reference.

In some cases, the reference may comprise a derivative of the reference. In some cases, the reference may comprise a result of the method performed on a reference sample. In some cases, the reference sample may be of a subject responsive to the treatment. In some cases, the comparing may be performed by a computer processor. In some cases, the comparing may be performed by a trained algorithm. In some cases, the reference may comprise a result obtained from genetic material of a subject diagnosed with endometriosis. In some cases, the reference may comprise a result obtained from genetic material of a subject responsive to the treatment.

In some cases, the method may further comprise detecting an epigenetic marker in at least a portion of the genetic material. In some cases, the epigenetic marker may comprise a methylated marker, a hydroxymethylated marker, a carboxylated marker, a formylated marker, or any combination thereof. In some cases, the portion may comprise the epigenetic marker is RNA or DNA.

In some cases, the method may further comprise reporting a result of the method. In some cases, the result may comprise an output of the detecting. In some cases, the reporting may comprise electronic reporting.

In some cases, the method may further comprise identifying the subject as a responder to the leuprolide acetate, the derivative thereof, the biosimilar thereof, or the interchangeable thereof. In some cases, the method may further comprise identifying the subject as a non-responder to the leuprolide acetate. In some cases, the identifying may be based in part on: a disease activity score; a presence, an absence, or a recurrence of pelvic pain; a cessation of the treatment; a scoring of dysmenorrhea; a presence of dyspareunia; a failure to conceive; a recurrence of a symptom following a treatment; a surgical intervention; or any combination thereof. In some cases, the identifying may be based on the presence, the absence, or the recurrence of pelvic pain. In some cases, the presence, the absence or the recurrence of pelvic pain may be reported by the subject on a visual analog scale (VAS). In some cases, the presence, the absence or the recurrence of pelvic pain may be reported after the treatment is completed. In some cases, the pelvic pain may comprise non-menstrual pelvic pain. In some cases, the identifying may be based on the disease activity score. In some cases, the identifying may be based at least in part on a medical history of the subject, a hormone receptor level of the subject, a mood of the subject, or any combination thereof. In some cases, the subject may be identified as the responder with a sensitivity of at least about 80%. In some cases, the subject may be identified as the responder with a specificity of at least about 80%. In some cases, the subject may be identified as the non-responder with a sensitivity of at least about 80%. In some cases, the subject may be identified as the non-responder with a specificity of at least about 80%.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a background of pharmacogenetics showing responders, non-responders, and toxic responders to a pharmaceutical composition.

FIG. 2 shows an example of differential metabolism to codeine.

FIG. 3 shows an exemplary structure of leuprolide acetate.

FIG. 4 shows an exemplary sensitivity v. specificity curve of genetic risk scores.

FIG. 5 shows a table of variants with significant correlated with failure or success of leuprolide acetate treatment. All had p<0.005 comparing responders to non-responders.

FIG. 6 shows a computer control system that is programmed or otherwise configured to implement methods provided herein.

FIG. 7 is a diagram showing a method and system as disclosed herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

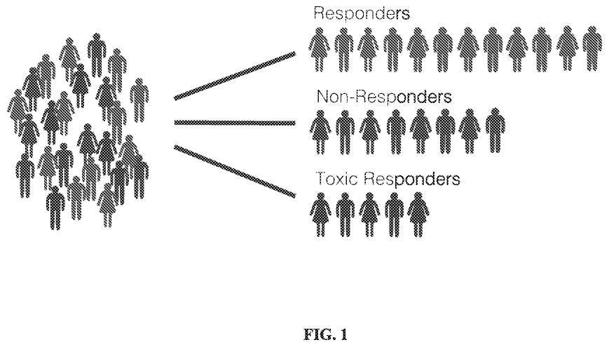

Referring to FIG. 1, plasma levels of an administered pharmaceutical composition may vary 1,000 fold in subjects taking identical doses of the pharmaceutical composition. These differences may be due in part to inherited differences in drug metabolic pathways that can affect an individual subject's response to the pharmaceutical composition or due in part to the presence of one or more genetic variants (such as a variant that may affect the expression of a disease) that can affect the response to a given treatment.

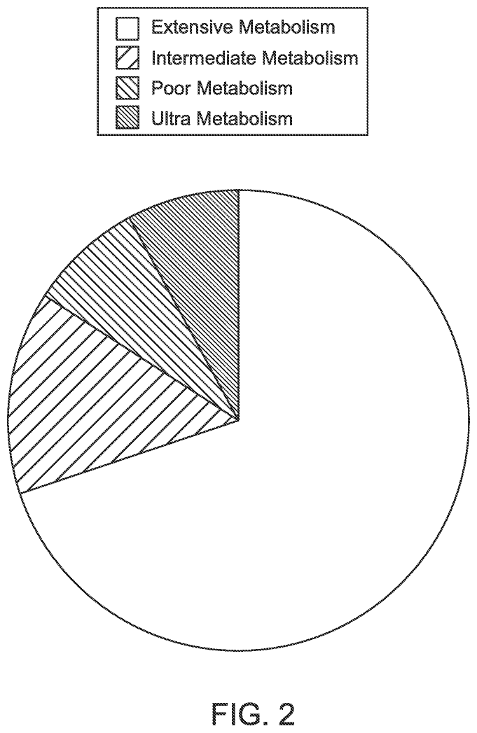

Referring to FIG. 2, one or more genetic variations may lead to differential responses to pain relief from codeine. In this example, codeine is an inactive “pro-drug” that is metabolized into an active form (morphine) by CYP2D6. Genetic variations in CYP2D6 may be poor metabolizers. Subjects having a reduced function or loss-of-function allele in CYP2D6 may receive reduced or no therapeutic pain relief from codeine. In some cases, a subject may receive no therapeutic pain relief from codeine and side effects from the treatment. Ultra-rapid metabolizers, such as those subjects having an increased gene copy number (up to 13) alleles have a high risk for morphine toxicity, respiratory depression, and death. Genetic variations causing variations in metabolism may produce a range of subject, each receiving a similar dose of codeine, but with a wide range of responses to the treatment from pain relief, to reduced pain relief, to no pain relief, to toxic side effects.

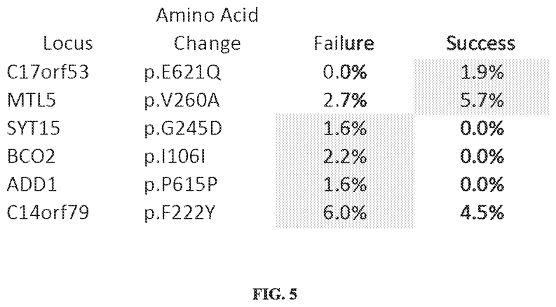

Leuprolide acetate may be prescribed to treat endometriosis. However, a significant number of subjects may have little or no improvement with LA therapy. For example, subjects may not receive reduced disease activity score, reduced pelvic pain, increase fertility, or any combination thereof. Identifying subjects that may respond to LA therapy may provide a significant improvement. Identifying subjects that may respond to LA therapy may be based at least in part on identifying a presence or an absence of one or more genetic variants in genetic material of the subject (such as one or more variants of Table 1 or Table 2, one or more genetic variants of a gene in FIG. 5, or a combination thereof). Identification of a presence of an absence of one or more genetic variants may also diagnosis or confirm diagnosis of endometriosis.

Methods as described herein may include detecting a presence or an absence of a genetic variant in genetic material obtained from a subject. A treatment may be selected based at least in part on a result of the detecting. Methods may include identifying a genetic variant or a panel of genetic variants that may be predictive of a subject responding or not responding to a treatment for a disease or condition. A genetic variant may include one or more genetic variants of Table 1 or Table 2 or a variant of a gene of FIG. 5. The disease or condition may be endometriosis or a related reproductive condition, such as non-menstrual pelvic pain. A treatment may include a pharmaceutical composition comprising leuprolide acetate. A treatment may include a combination of more than one active ingredient. Methods as described herein may include obtaining a sample from a subject, isolating genetic material from a sample, treating the subject with a treatment, recommending a treatment to the subject, comparing a result of the detecting with a reference, inputting a result of the detecting into a trained algorithm, or any combination thereof. Methods may identify with at least about 80% sensitivity, with at least about 80% specificity, with at least about 80% accuracy, or any combination thereof, a subject's responsiveness to a treatment.

Methods as described herein may identify a subject as a responder or a non-responder to a treatment for endometriosis or pain. The treatment may be administration of leuprolide acetate, a derivative thereof, a biosimilar thereof, or an interchangeable thereof. The identifying may include detecting a presence or an absence of one or more genetic variants in a genetic material of the subject.

### Definitions

Unless otherwise indicated, open terms for example “contain,” “containing,” “include,” “including,” and the like mean comprising.

The singular forms “a”, “an”, and “the” are used herein to include plural references unless the context clearly dictates otherwise. Accordingly, unless the contrary is indicated, the numerical parameters set forth in this application are approximations that may vary depending upon the desired properties sought to be obtained by the present invention.

Unless otherwise indicated, some instances herein contemplate numerical ranges. When a numerical range is provided, unless otherwise indicated, the range includes the range endpoints. Unless otherwise indicated, numerical ranges include all values and subranges therein as if explicitly written out. Unless otherwise indicated, any numerical ranges and/or values herein, following or not following the term “about,” can be at 85-115% (i.e., plus or minus 15%) of the numerical ranges and/or values.

As used herein, “endometriosis” can refer to any nonmalignant disorder in which functioning endometrial tissue is present in a location in the body other than the endometrium of the uterus, i.e. outside the uterine cavity or is present within the myometrium of the uterus. For purposes herein it also includes conditions, such as adenomyosis/adenomyoma, that exhibit myometrial tissue in the lesions. Endometriosis can include endometriosis externa, endometrioma, adenomyosis, adenomyomas, adenomyotic nodules of the uterosacral ligaments, endometriotic nodules other than of the uterosacral ligaments, autoimmune endometriosis, mild endometriosis, moderate endometriosis, severe endometriosis, superficial (peritoneal) endometriosis, deep (invasive) endometriosis, ovarian endometriosis, endometriosis-related cancers, and/or “endometriosis-associated conditions”. Unless stated otherwise, the term endometriosis is used herein to describe any of these conditions.

As used herein, “treating” can include one or more of: reducing the frequency and/or severity of symptoms (such as pelvic pain), elimination of symptoms and/or their underlying cause, and improvement or remediation of damage. For example, treatment of endometriosis includes, for example, relieving the pain experienced by a woman suffering from endometriosis, and/or causing the regression or disappearance of endometriotic lesions. Treating may also include: improved fertility or ability to conceive, cessation of dyspareunia, absence of non-menstrual pelvic pain, or any combination thereof.

Biological samples obtained from individuals (e.g., human subjects) may be any sample from which a genetic material (e.g., nucleic acid sample) may be derived. Genetic material may be obtained from endometrial tissue. Genetic material may be obtained from any reproductive tissue (such as endometrial tissue, ovarian tissue, fallopian tissue, cervical tissue, vulvar tissue, uterine tissue, or any combination thereof. Samples/Genetic materials may be from tissue, tissue biopsy, liquid biopsy, fine needle aspirate, buccal swab, saliva, blood, hair, nail, skin, cell, or any other type of tissue sample. In some instances, the genetic material (e.g., nucleic acid sample) comprises mRNA, cDNA, genomic DNA, or PCR amplified products produced therefrom, or any combination thereof. In some instances, the genetic material (e.g., nucleic acid sample) comprises PCR amplified nucleic acids produced from cDNA or mRNA. In some instances, the genetic material (e.g., nucleic acid sample) comprises PCR amplified nucleic acids produced from genomic DNA.

As used herein, the term “cell-free” or “cell free” can refer to the condition of the nucleic acid sequence as it appeared in the body before the sample is obtained from the body. For example, circulating cell-free nucleic acid sequences in a sample may have originated as cell-free nucleic acid sequences circulating in the bloodstream of the human body. In contrast, nucleic acid sequences that are extracted from a solid tissue, such as a biopsy, are generally not considered to be “cell-free.” In some cases, cell free nucleic acids can include cell free DNA or cell free RNA. In some cases, cell-free DNA may comprise fetal DNA, maternal DNA, or a combination thereof. In some cases, cell-free DNA may comprise DNA fragments released into a blood plasma. In some cases, the cell-free DNA may comprise circulating tumor DNA. In some cases, cell-free DNA may comprise circulating DNA indicative of a tissue origin, a disease or a condition. A cell-free nucleic acid sequence may be isolated from a blood sample. A cell-free nucleic acid sequence may be isolated from a plasma sample, urine, saliva, or synovial fluids. A cell-free nucleic acid sequence may comprise a complementary DNA (cDNA). In some cases, one or more cDNAs may form a cDNA library.

The term “epigenetic modification” as used herein, may be any covalent modification of a nucleic acid base. In some cases, a covalent modification may comprise (i) adding a methyl group, a hydroxymethyl group, a carbon atom, an oxygen atom, or any combination thereof to one or more bases of a nucleic acid sequence, (ii) changing an oxidation state of a molecule associated with a nucleic acid sequence, such as an oxygen atom, or (iii) a combination thereof. A covalent modification may occur at any base, such as a cytosine, a thymine, an uracil, an adenine, a guanine, or any combination thereof. In some cases, an epigenetic modification may comprise an oxidation or a reduction. A nucleic acid sequence may comprise one or more epigenetically modified bases. An epigenetically modified base may comprise any base, such as a cytosine, an uracil, a thymine, adenine, or a guanine. An epigenetically modified base may comprise a methylated base, a hydroxymethylated base, a formylated base, or a carboxylic acid containing base or a salt thereof. An epigenetically modified base may comprise a 5-methylated base, such as a 5-methylated cytosine (5-mC). An epigenetically modified base may comprise a 5-hydroxymethylated base, such as a 5-hydroxymethylated cytosine (5-hmC). An epigenetically modified base may comprise a 5-formylated base, such as a 5-formylated cytosine (5-fC). An epigenetically modified base may comprise a 5-carboxylated base or a salt thereof, such as a 5-carboxylated cytosine (5-caC). In some cases, an epigenetically modified base may comprise a methyltransferase-directed transfer of an activated group (mTAG). An epigenetic modification may also include a nucleic acid and or protein modifications which may include but is not limited to: citrullination, glycosylation, phosphorylation, acetylation, methylation (examples—5-mc, 5-hmc, 5-fc, 5-caC, 5-hmU, 6-mA (6-methyladenine), N4-methylcytosine), myristoylation, ubiquitylation, sumoylation, ribosylation, prenylation, or any combination thereof.

An epigenetically modified base may comprise one or more bases or a purine (such as Structure 1) or one or more bases of a pyrimidine (such as Structure 2). An epigenetic modification may occur one or more of any positions. For example, an epigenetic modification may occur at one or more positions of a purine, including positions 1, 2, 3, 4, 5, 6, 7, 8, 9, as shown in Structure 1. In some cases, an epigenetic modification may occur at one or more positions of a pyrimidine, including positions 1, 2, 3, 4, 5, 6, as shown in Structure 2.

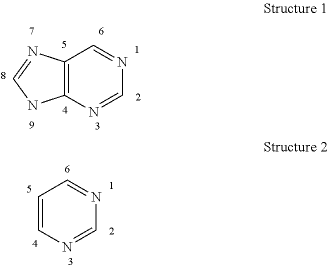 [SMILES: c1ncc2nc[nH]c2n1 ; c1cncnc1]

A nucleic acid sequence may comprise an epigenetically modified base. A nucleic acid sequence may comprise a plurality of epigenetically modified bases. A nucleic acid sequence may comprise an epigenetically modified base positioned within a CG site, a CpG island, or a combination thereof. A nucleic acid sequence may comprise different epigenetically modified bases, such as a methylated base, a hydroxymethylated base, a formylated base, a carboxylic acid containing base or a salt thereof, a plurality of any of these, or any combination thereof.

The term “subject,” as used herein, may be any animal or living organism. Animals can be mammals, such as humans, non-human primates, rodents such as mice and rats, dogs, cats, pigs, sheep, rabbits, and others. A subject may be a dog. A subject may be a human. Animals can be fish, reptiles, or others. Animals can be neonatal, infant, adolescent, or adult animals. Humans can be more than about: 1, 2, 5, 10, 20, 30, 40, 50, 60, 65, 70, 75, or at least about 80 years of age. The subject may have or be suspected of having a condition or a disease, such as endometriosis or related condition. The subject may be a patient, such as a patient being treated for a condition or a disease, such as a patient suffering from endometriosis. The subject may be predisposed to a risk of developing a condition or a disease such as endometriosis. The subject may be in remission from a condition or a disease, such as a patient recovering from endometriosis. The subject may be healthy. The subject may be a subject in need thereof. The subject may be a female subject or a male subject.

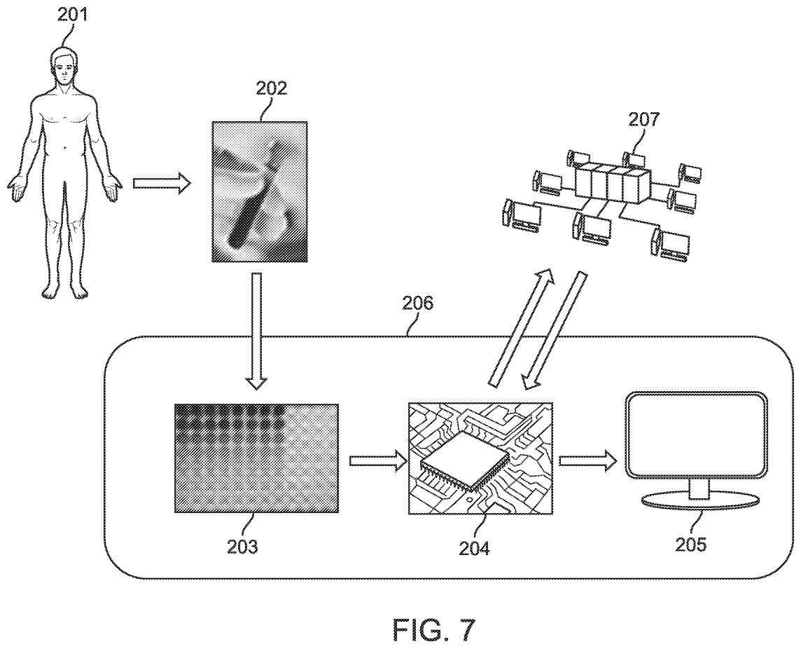

A sample comprising genetic material may be obtained from a subject, such as a subject in need thereof. As shown in FIG. 7, a sample 202 containing a genetic material may be obtained from a subject 201, such as a human subject. A sample 202 may be subjected to one or more methods as described herein, such as performing an assay. In some cases, an assay may comprise sequencing, genotyping, hybridization, amplification, labeling, or any combination thereof. One or more results from a method may be input into a processor 204. One or more input parameters such as a sample identification, subject identification, sample type, a reference, or other information may be input into a processor 204. One or more metrics from an assay may be input into a processor 204 such that the processor may produce a result, such as a diagnosis of endometriosis, a recommendation for treatment, or a combination thereof. A processor may send a result, an input parameter, a metric, a reference, or any combination thereof to a display 205, such as a visual display or graphical user interface. A processor 204 may (i) send a result, an input parameter, a metric, or any combination thereof to a server 207, (ii) receive a result, an input parameter, a metric, or any combination thereof from a server 207, (iii) or a combination thereof.

The term “sequencing” as used herein, may comprise high-throughput sequencing, next-gen sequencing, Maxam-Gilbert sequencing, massively parallel signature sequencing, Polony sequencing, 454 pyrosequencing, pH sequencing, Sanger sequencing (chain termination), Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, Enigma sequencing, sequencing-by-hybridization, sequencing-by-ligation, or any combination thereof. The sequencing output data may be subject to quality controls, including filtering for quality (e.g., confidence) of base reads. Exemplary sequencing systems include 454 pyrosequencing (454 Life Sciences), Illumina (Solexa) sequencing, SOLiD (Applied Biosystems), and Ion Torrent Systems' pH sequencing system. In some cases, a nucleic acid of a sample may be sequenced without an associated label or tag. In some cases, a nucleic acid of a sample may be sequenced, the nucleic acid of which may have a label or tag associated with it.

“Haplotype” can mean a combination of genotypes on the same chromosome occurring in a linkage disequilibrium block. Haplotypes serve as markers for linkage disequilibrium blocks, and at the same time provide information about the arrangement of genotypes within the blocks. Typing of only certain variants which serve as tags can, therefore, reveal all genotypes for variants located within a block. Thus, the use of haplotypes greatly facilitates identification of candidate genes associated with diseases and drug sensitivity.

“Linkage disequilibrium” or “LD” can mean that a particular combination of alleles (alternative nucleotides) or genetic variants for example at two or more different SNP (or RV) sites are non-randomly co-inherited (i.e., the combination of alleles at the different SNP (or RV) sites occurs more or less frequently in a population than the separate frequencies of occurrence of each allele or the frequency of a random formation of haplotypes from alleles in a given population). The term “LD” can differ from “linkage,” which describes the association of two or more loci on a chromosome with limited recombination between them. LD can also be used to refer to any non-random genetic association between allele(s) at two or more different SNP (or RV) sites. In some instances, when a genetic marker (e.g. SNP or RV) is identified as the genetic marker associated with a responsiveness to a treatment for a disease (in this instance endometriosis), it can be the minor allele (MA) of the particular genetic marker that is associated with the responsiveness. In some instances, if the Odds Ratio (OR) of the MA is greater than 1.0, the MA of the genetic marker (in this instance the endometriosis associated genetic marker) can be correlated with an increased probability of a subject responding to a treatment as compared to a control subject, and if the OR of the MA less than 1.0, the MA of the genetic marker can be correlated with a decreased probability of a subject responding to a treatment as compared to a control subject. “Linkage disequilibrium block” or “LD block” can mean a region of the genome that contains multiple variants located in proximity to each other and that are transmitted as a block.

As used herein, a “biosimilar” or a “biosimilar product” may refer to a biological product that is licensed based on a showing that it is substantially similar to an FDA-approved biological product, known as a reference product, and has no clinically meaningful differences in terms of safety and effectiveness from the reference product. Only minor differences in clinically inactive components may be allowable in biosimilar products. A “biosimilar” of an approved reference product/biological drug refers to a biologic product that is similar to the reference product based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biological product. In some embodiments, the biosimilar biological product and reference product utilize the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biological product have been previously approved for the reference product. In some embodiments, the route of administration, the dosage form, and/or the strength of the biological product are the same as those of the reference product. In some embodiments, the facility in which the biological product is manufactured, processed, packed, or held may meet standards designed to assure that the biological product continues to be safe, pure, and potent. The reference product may be approved in at least one of the U.S., Europe, or Japan. In some embodiments, a response rate of human subjects administered the biosimilar product can be 50%-150% of the response rate of human subjects administered the reference product. For example, the response rate of human subjects administered the biosimilar product can be 50%-100%, 50%-110%, 50%-120%, 50%-130%, 50%-140%, 50%-150%, 60%-100%, 60%-110%, 60%-120%, 60%-130%, 60%-140%, 60%-150%, 70%-100%, 70%-110%, 70%-120%, 70%-130%, 70%-140%, 70%-150%, 80%-100%, 80%-110%, 80%-120%, 80%-130%, 80%-140%, 80%-150%, 90%-100%, 90%-110%, 90%-120%, 90%-130%, 90%-140%, 90%-150%, 100%-110%, 100%-120%, 100%-130%, 100%-140%, 100%-150%, 110%-120%, 110%-130%, 110%-140%, 110%-150%, 120%-130%, 120%-140%, 120%-150%, 130%-140%, 130%-150%, or 140%-150% of the response rate of human subjects administered the reference product. In some embodiments, a biosimilar product and a reference product can utilize the same mechanism or mechanisms of action for the condition or conditions of use prescribed, recommended, or suggested in the proposed labeling, but only to extent the mechanism or mechanisms are known for the reference product. To obtain approval for biosimilar drugs, studies and data of structure, function, animal toxicity, pharmacokinetics, pharmacodynamics, immunogenicity, and clinical safety and efficacy may be needed. A biosimilar may also be known as a follow-on biologic or a subsequent entry biologic. In some embodiments, a biosimilar product may be substantially similar to the reference product notwithstanding minor different in clinically inactive components.

As used herein, a “interchangeable biological product” may refer to a biosimilar of an FDA-approved reference product and may meet additional standards for interchangeability. In some embodiments, an interchangeable biological product can, for example, produce the same clinical result as the reference product in any given subject. In some embodiments, an interchangeable product may contain the same amount of the same active ingredients, may possess comparable pharmacokinetic properties, may have the same clinically significant characteristics, and may be administered in the same way as the reference compound. In some embodiments, an interchangeable product can be a biosimilar product that meets additional standards for interchangeability. In some embodiments, an interchangeable product can produce the same clinical result as a reference product in all the reference product's licensed conditions of use. In some embodiments, an interchangeable product can be substituted for the reference product by a pharmacist without the intervention of the health care provider who prescribed the reference product. In some embodiments, when administered more than once to an individual, the risk in terms of safety or diminished efficacy of alternating or switching between use of the biological product and the reference product is not greater than the risk of using the reference product without such alternation or switch. In some embodiments, an interchangeable product can be a regulatory agency approved product. In some embodiments, a response rate of human subjects administered the interchangeable product can be 80%-120% of the response rate of human subjects administered the reference product. For example, the response rate of human subjects administered the interchangeable product can be 80%-100%, 80%-110%, 80%-120%, 90%-100%, 90%-110%, 90%-120%, 100%-110%, 100%-120%, or 110%-120 of the response rate of human subjects administered the reference product.

A treatment may comprise a receptor agonist. A receptor agonist may be a full agonist, a co-agonist, a selective agonist, a partial agonist, an inverse agonist, a super-agonist, an irreversible agonist, or any combination thereof. A treatment may comprise a receptor antagonist. A receptor antagonist may be a competitive antagonist, a non-competitive antagonist, an uncompetitive antagonist, a silent antagonist, a partial agonist that may act as a competitive antagonist, an inverse agonist that can act as an antagonist, or any combination thereof. A treatment may comprise a mixed agonist/antagonist.

### Identifying a Treatment

A subject may respond to a treatment. A subject may not respond to a treatment. Methods as described herein may identify or predict subjects that may respond to treatment, identify or predict subjects that may not respond to treatment, or a combination thereof. Identifying or predicting a subject that responds to a treatment may be based at least in part on one or more differences in genetic material obtained from the subject. For example, a genetic variant or panel of genetic variants may identify a subject as responsive to a treatment or non-responsive to a treatment. Selection of a treatment from a plurality of treatments may be based at least in part on a result of a genetic analysis performed on a sample from the subject. A treatment may be an FDA-approved treatment for endometriosis. A treatment may be a hormone-based treatment, a biosimilar thereof or an interchangeable thereof. A treatment may be a leuprolide acetate, a derivative thereof, a biosimilar thereof, an interchangeable thereof, or a salt thereof.

Identifying a treatment for a subject may comprise detecting a presence or an absence of a genetic variant in genetic material from a subject. A genetic variant may comprise a single nucleotide polymorphism (SNP). A genetic variant may comprise a variation in copy number. A genetic variant may comprise a genetic mutation. A genetic variant can comprise a synonymous mutation, a non-synonymous mutation, a stop-gain mutation, a nonsense mutation, an insertion, a deletion, a splice-site variant, a frameshift mutation, or any combination thereof. A genetic variant can comprise a protein damaging mutation. A genetic variant may be a rare variant occurring in less than about: 1%, 0.5%, 0.1%, 0.05% or 0.01% of a population. A genetic variant may have a minor allele frequency (MAF) of less than about 1% of a population. A genetic variant may be selected from Table 1 or Table 2. A genetic variant may be of a gene listed in FIG. 5. More than one genetic variant may be detected, such as about 2, 3, 4, 5, 6, 7, 8, 9, 10 genetic variants or more. A presence of one or more genetic variants in Table 1 in a genetic material of a subject may identify the subject as responsive to a treatment (such as leuprolide acetate). A presence of one or more genetic variants in Table 2 in a genetic material of a subject may identify the subject as non-responsive to a treatment (such as a treatment comprising leuprolide acetate). A presence of one or more genetic variants in Table 2 in a genetic material of a subject may identify the subject as responsive to a treatment, such as a treatment comprising leuprolide acetate and a second active ingredient such as a combination therapy).

Detecting a presence of a genetic variant may be at least in part indicative of a therapeutically effective response to a particular treatment. Detecting a presence of a genetic variant may be at least in part indicative of a lack of therapeutically effective response to a particular treatment. In some cases, detecting a presence of one or more genetic variants of Table 1 may be at least in part indicative of a therapeutically effective response to a treatment comprising leuprolide acetate, a derivative thereof, a biosimilar thereof, or an interchangeable thereof. In some cases, detecting a presence of one or more genetic variants of Table 2 may be at least in part indicative of a lack in therapeutically effective response to a treatment comprising leuprolide acetate. In some cases, a subject may receive a treatment comprising leuprolide acetate and upon detecting one or more genetic variants (such as one or more of Table 2), the treatment may be altered to one that does not comprise leuprolide acetate. In some cases, a subject may receive a treatment comprising leuprolide acetate and upon detecting one or more genetic variants (such as one or more of Table 1), the subject may continue to receive the treatment.

A method may comprise detecting one or more variants of Table 1. In some cases, detecting may comprise detecting one or more of variants #1, #4-#7, #9-#11, #18, or any combination thereof of Table 1. Detecting may comprise detecting variant #1 of Table 1. Detecting may comprise detecting variant #4 of Table 1. Detecting may comprise detecting variant #5 of Table 1. Detecting may comprise detecting variant #6 of Table 1. Detecting may comprise detecting variant #7 of Table 1. Detecting may comprise detecting variant #9 of Table 1. Detecting may comprise detecting variant #10 of Table 1. Detecting may comprise detecting variant #11 of Table 1. Detecting may comprise detecting variant #18 of Table 1. In some cases, a presence of the variant in genetic material of a subject may be indicative that the subject will respond to a treatment for endometriosis (such as leuprolide acetate). In some cases, a combination of the variants may be selection as a panel of variants for detection in a genetic material obtained from a subject.

A method may comprise detecting one or more variants of Table 2. In some cases, detecting may comprise detecting one or more of variants #19-424, #26, #27, #31, #33-#38, #41-#45, #47, or any combination thereof of Table 2. Detecting may comprise detecting variant #19 of Table 2. Detecting may comprise detecting variant #20 of Table 2. Detecting may comprise detecting variant #21 of Table 2. Detecting may comprise detecting variant #22 of Table 2. Detecting may comprise detecting variant #23 of Table 2. Detecting may comprise detecting variant #24 of Table 2. Detecting may comprise detecting variant #26 of Table 2. Detecting may comprise detecting variant #27 of Table 2. Detecting may comprise detecting variant #31 of Table 2. Detecting may comprise detecting variant #33 of Table 2. Detecting may comprise detecting variant #34 of Table 2. Detecting may comprise detecting variant #35 of Table 2. Detecting may comprise detecting variant #36 of Table 2. Detecting may comprise detecting variant #37 of Table 2. Detecting may comprise detecting variant #38 of Table 2. Detecting may comprise detecting variant #41 of Table 2. Detecting may comprise detecting variant #42 of Table 2. Detecting may comprise detecting variant #43 of Table 2. Detecting may comprise detecting variant #44 of Table 2. Detecting may comprise detecting variant #45 of Table 2. Detecting may comprise detecting variant #47 of Table 2. In some cases, a presence of the variant in genetic material of a subject may be indicative that the subject will respond to a treatment for endometriosis (a treatment that is not leuprolide acetate or a derivative thereof). In some cases, a combination of the variants may be selection as a panel of variants for detection in a genetic material obtained from a subject.

### Responsiveness to Treatment

Responsiveness to a treatment may be based on one or more factors including: (i) a presence or an absence of a genetic variant in a sample from the subject; (ii) a disease activity score; (iii) a presence, an absence, or a recurrence of pelvic pain (such as non-menstrual pelvic pain); (iv) a cessation of the treatment prior to completion; (v) a scoring of dysmenorrhea severity; (vi) a presence of dyspareunia following treatment; (vii) a failure to conceive following treatment; (viii) a recurrence of a symptom following treatment; (ix) a surgical intervention following treatment, or any combination thereof. In some cases, a failed treatment may be one in which: (a) a subject may not tolerate the treatment and may terminate the treatment before completion; (b) a subject experiences a recurrence of pelvic pain after treatment; (c) a subject receives a surgical intervention after treatment; (d) a subject fails to conceive after treatment; (e) a subject suffers from dyspareunia after treatment; (f) a subject receives a disease activity score indicating a presence of endometriosis after treatment; (g) or any combination thereof. In some cases, a successful treatment may be one in which: (a) a subject does not experience pelvic pain following treatment; (b) a subject conceives after treatment; (c) a subject receives a disease activity score indicating an absence of endometriosis; (d) or any combination thereof. A disease activity score may be administered by a medical professional. A pelvic pain may be a non-menstrual pelvic pain. A pelvic pain may be self-reported by the subject. A pelvic pain may be reported on a visual analog scale (VAS).

### Treatments

A treatment may be selected based at least in part on genetic information of the subject. As described herein, a presence or an absence of one or more genetic variants in a genetic material obtained from a subject may inform the selection of treatment, such as a treatment for endometriosis. The selection of the treatment may occur after or based at least in part on assessing a mood of the subject, measuring one or more hormone levels of the subject, reviewing a medical history of the subject, or any combination thereof.

A treatment can comprise administration of a composition. A composition can be a pharmaceutical composition. A composition can be an FDA-approved treatment for endometriosis. A treatment can comprise an off-label use of a pharmaceutical composition. A treatment can comprise a hormone treatment, or treatment with a biosimilar. A composition can comprise leuprolide acetate, a derivative thereof, a biosimilar thereof, an interchangeable thereof, or a salt thereof. A composition can comprise a synthetic analog of a gonadotropin releasing hormone (GnRH), a derivative thereof, a biosimilar thereof, or an interchangeable thereof. A composition can comprise a GnRH receptor agonist, a GnRH receptor antagonist, a progestin, a progesterone, an estrogen, a norethindrone, a medroxyprogesterone, a salt of any of these, a biosimilar of any of these, an interchangeable of any of these, or any combination thereof. A composition can comprise Mifepristone® (RU-486, CAS #84371-65-3), Gestrinone® (Ethylnorgestrienone, CAS #16320-04-0), Danazol® (2,3-Isoxazolethisterone, CAS #17230-88-5), Orilissa® (elagolix, CAS #834153-87-6), Zoladex® (Goserelin, CAS #65807-02-5), Aygestin® (Norethindrone acetate, CAS #38673-38-0), Depo-Provera® (Methylhydroxyprogesterone acetate, CAS #71-58-9), a salt of any of these, a biosimilar of any of these, an interchangeable or any of these, or any combination thereof.

A pharmaceutical composition can comprise a first active ingredient. The first active ingredient can comprise a treatment for endometriosis (such as leuprolide acetate). The pharmaceutical composition can be formulated in unit dose form. The pharmaceutical composition can comprise a pharmaceutically acceptable excipient, diluent, or carrier. The pharmaceutical composition can comprise a second, third, or fourth active ingredient. A second ingredient may be an add-back component (such as norethindrone acetate, a biosimilar, an interchangeable thereof, or a salt thereof).

A method of treating endometriosis may comprise administering a hormonal therapy to a subject. In some instances, the hormone can be progestin, progestogen, progesterone, desogestrel, etonogestrel, gestodene, levonorgestrel, medroxyprogesterone, norethisterone, norgestimate, megestrol, megestrol acetate, norgestrel, a pharmaceutically acceptable salt thereof (e.g., acetate), or any combination thereof. In some instances, a therapeutic used herein is selected from progestins, estrogens, antiestrogens, and antiprogestins, for example micronized danazol in a micro- or nanoparticulate formulation. Methods and therapeutics presented herein can utilize an active agent in a freebase, salt, hydrate, polymorph, isomer, diastereomer, prodrug, metabolite, ion pair complex, or chelate form. An active agent can be formed using a pharmaceutically acceptable non-toxic acid or base, including an inorganic acid or base, or an organic acid or base. In some instances, an active agent that can be utilized in connection with the methods and compositions presented herein is a pharmaceutically acceptable salt derived from acids including, but not limited to, the following: acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, or p-toluenesulfonic acid. For further description of pharmaceutically acceptable salts that can be used in the methods described herein see, for example, S. M. Barge et al., “Pharmaceutical Salts,” 1977, J. Pharm. Sci. 66:1-19, which is incorporated herein by reference in its entirety.

In some instances, the therapeutic may take the form of a testosterone or a modified testosterone such as Danazol®. In some instances, the therapeutic can be a hormonal treatment therapeutic which may be administered alone or in combination with a gene therapy. For instance, the therapeutic may be an estrogen containing composition, a progesterone containing composition, a progestin containing composition, a gonadotropin releasing-hormone (GnRH) receptor agonist, a gonadotropin releasing-hormone (GnRH) receptor antagonist, or other ovulation suppression composition, or a combination thereof. In some instances, the GnRH receptor agonist may take the form of a GnRH receptor agonist in combination with a patient specific substantially low dose of estrogen, progestin, or tibolone via an add-back administration. In some instances, in such add-back therapy, the dosage of estrogen, progestin, or tibolone may be relatively small to not reduce the effectiveness of the GnRH receptor agonist. In some instances, the therapeutic is an oral contraceptive (OC). In some instances, the OC is in a pill form that is comprised at least partially of estrogen, progesterone, or a combination thereof. In some instances, the progesterone component may be any of Desogestrel, Drospirenone, Ethynodiol, Levonorgestrel, Norethindrone, Norgestimate, and Norgestrel, and the estrogen component may further be any of Mestranol, Estradiol, and Ethinyl. In some instances, the OC may be any commercially available OC including ALESSE, APRI, ARANELLE, AVIANE, BREVICON, CAMILA, CESIA, CRYSELLE, CYCLESSA, DEMULEN, DESOGEN, ENPRESSE, ERRIN, ESTROSTEP, JOLIVETTE, JUNEL, KARIVA, LEENA, LESSINA, LEVLEN, LEVORA, LOESTRIN, LUTERA, MICROGESTIN, MICRONOR, MIRCETTE, MODICON, MONONESSA, NECON, NORA, NORDETTE, NORINYL, NOR-QD, NORTREL, OGESTREL, ORTHO-CEPT, ORTHO-CYCLEN, ORTHO-NOVUM, ORTHO-TRI-CYCLEN, OVCON, OVRAL, OVRETTE, PORTIA, PREVIFEM, RECLIPSEN, SOLIA, SPRINTEC, TRINESSA, TRI-NORINYL, TRIPHASIL, TRIVORA, VELIVET, YASMIN, AND ZOVIA (the preceding names are the registered trademarks of the respective providers).

A progestin may include any of a first-generation progestin (estrane) including norethindrone, norethynodrel, norethindrone acetate, or ethynodiol diacetate; a second-generation progestin (gonane) including levonorgestrel, norethisterone, or norgestrel; a third-generation progestin (gonane) including desogestrel, gestodene, norgestimate, drospirenone; a fourth-generation progestin including dienogest, nestorone, nomegestrol acetate, trimegestone; or any combination thereof. A progesterone may include tanaproget.

Cannabis may be preferably free of a THC portion. Cannabis may include a derivative thereof or may include a nabilone, a dronabinol, a nabiximol, or any combination thereof. An NSAID any salicylate. An NSAID may include aspirin (acetylsalicylic acid), diflunisal, salsalate, a propionic acid derivative (including ibuprofen, dexibuprofen, naproxen, fenoprofen, ketoprofen, dexketoprofen, flurbiprofen, oxaprozin, or loxoprofen), an acetic acid derivative (including indomethacin, tolmetin, sulindac, etodolac, ketorolac, diclofenac, or nabumetone), an enolic acid (oxicam) derivative (including piroxicam, meloxicam, tenoxicam, droxicam, lornoxicam, or isoxicam), a fenamic acid derivative (fenamates) (including efenamic acid, meclofenamic acid, flufenamic acid, or tolfenamic acid), a selective COX-2 inhibitor (Coxibs) (including celecoxib, rofecoxib, valdecoxib, parecoxib, lumiracoxib, etoricoxib, or firocoxib), a sulphonanilide (including nimesulide), licofelone (acts by inhibiting LOX (lipooxygenase) & COX and hence known as LOX/COX inhibitor) or lysine clonixinate; a natural NSAID (including hyperforin, figwort, or calcitriol (vitamin D)); or any combination thereof. A composition or treatment may comprise human serum albumin, such as AMPION®.

### Suitable Excipients

A composition described herein can compromise an excipient. An excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a trigylceride, an alcohol, or any combination thereof. An excipient can comprise sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerine, xanthan rubber, soy isoflavones, polysorbate 80, ethyl alcohol, water, teprenone, or any combination thereof. An excipient can be an excipient described in the Handbook of Pharmaceutical Excipients, American Pharmaceutical Association (1986).

Non-limiting examples of suitable excipients can include a buffering agent, a preservative, a stabilizer, a binder, a compaction agent, a lubricant, a chelator, a dispersion enhancer, a disintegration agent, a flavoring agent, a sweetener, a coloring agent.

In some cases, an excipient can be a buffering agent. Non-limiting examples of suitable buffering agents can include sodium citrate, magnesium carbonate, magnesium bicarbonate, calcium carbonate, and calcium bicarbonate. As a buffering agent, sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts or combinations thereof can be used in a pharmaceutical formulation.

In some cases, an excipient can comprise a preservative. Non-limiting examples of suitable preservatives can include antioxidants, such as alpha-tocopherol and ascorbate, and antimicrobials, such as parabens, chlorobutanol, and phenol. Antioxidants can further include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some instances a preservatives can include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitor.

In some cases, a pharmaceutical formulation can comprise a binder as an excipient. Non-limiting examples of suitable binders can include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

The binders that can be used in a pharmaceutical formulation can be selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some cases, a pharmaceutical formulation can comprise a lubricant as an excipient. Non-limiting examples of suitable lubricants can include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil. The lubricants that can be used in a pharmaceutical formulation can be selected from metallic stearates (such as magnesium stearate, calcium stearate, aluminium stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some cases, a pharmaceutical formulation can comprise a dispersion enhancer as an excipient. Non-limiting examples of suitable dispersants can include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some cases, a pharmaceutical formulation can comprise a disintegrant as an excipient. In some cases, a disintegrant can be a non-effervescent disintegrant. Non-limiting examples of suitable non-effervescent disintegrants can include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some cases, a disintegrant can be an effervescent disintegrant. Non-limiting examples of suitable effervescent disintegrants can include sodium bicarbonate in combination with citric acid, and sodium bicarbonate in combination with tartaric acid.

In some cases, an excipient can comprise a flavoring agent. Flavoring agents incorporated into an outer layer can be chosen from synthetic flavor oils and flavoring aromatics; natural oils; extracts from plants, leaves, flowers, and fruits; and combinations thereof. In some cases, a flavoring agent can be selected from the group consisting of cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some cases, an excipient can comprise a sweetener. Non-limiting examples of suitable sweeteners can include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, sylitol, and the like.

A composition may comprise a combination of the active agent, (e.g., leuprolide acetate), and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

### Administration and Dosing

Administration of a treatment can be affected in one dose, continuously or intermittently throughout the course of treatment. Methods of determining the most effective means and dosage of administration are known to those of skill in the art and can vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and can vary with the composition used for treatment, the purpose of the treatment, the health condition or disease stage of the subject being treated, and target cell or tissue. Non-limiting examples of route of administration include oral administration, nasal administration, injection (such as intramuscular), and topical application.

Administration can refer to methods that can be used to enable delivery of a treatment. These methods can include topical administration (such as a lotion, a cream, an ointment) to an external surface of a surface, such as a skin. These methods can include parenteral administration (including intravenous, subcutaneous, intrathecal, intraperitoneal, intramuscular, intravascular or infusion), oral administration, inhalation administration, intraduodenal administration, rectal administration. In some instances, a subject can administer the treatment in the absence of supervision. In some instances, a subject can administer the treatment under the supervision of a medical professional (e.g., a physician, nurse, physician's assistant, orderly, hospice worker, etc.). In some cases, a medical professional can administer the treatment. In some cases, a cosmetic professional can administer the treatment. In some cases, the treatment is given to a subject by injection, such an intramuscular injection.

A dosing of a treatment may be about: 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, or 950 mg in a single dose. A dosing of a treatment may be from about 1 mg to about 20 mg. A dosing of a treatment may be from about 20 mg to about 50 mg. A dosing of a treatment may be from about 50 mg to about 100 mg. A dosing of a treatment may be from about 100 mg to about 200 mg. A dosing of a treatment may be from about 200 mg to about 300 mg. A dosing of a treatment may be from about 300 mg to about 400 mg. A dosing of a treatment may be from about 400 mg to about 500 mg. A dosing of a treatment may be from about 500 mg to about 600 mg. A dosing of a treatment may be adjusted based on one or more symptoms, disease severity, weight of a subject, or any combination thereof.

A dosing of a treatment may be about 3.75 mg, dosed up to 6 months. A dosing of a treatment may be about 11.25 mg, dosed every 3 months for 2 doses. In some cases, a dosing of a treatment may be dosed up to 6 months in total. A dosing of a treatment may be from about 2 mg to about 6 mg. A dosing of a treatment may be from about 3 mg to about 5 mg. A dosing of a treatment may be from about 10 to about 15 mg. A dosing of a treatment may be from about 8 mg to about 12 mg.

A dosing of a treatment may be about 600 mg, dosed in a single dose. A dosing of a treatment may be from about 500 mg to about 700 mg. A dosing of a treatment may be from about 550 mg to about 650 mg. A dosing of a treatment may be dosed in more than one dose. A dosing of a treatment may be dosed daily. A dosing of a treatment may be dosed bi-weekly.

A dosing of a treatment may be about 60 ug, dosed every three days. A dosing of a treatment may be from about 50 ug to about 70 ug, dosed every three days. A dosing of a treatment may be from about 55 ug to about 65 ug, dosed every three days.

A dosing of a treatment may be about 2.5 mg, dosed two times per week. A dosing of a treatment may be from about 2 mg to about 3 mg, dosed two times per week. A dosing of a treatment may be from about 1 mg to about 4 mg, dosed two times per week.

A dosing of a treatment may be about 5 mg, dosed daily. A dosing of a treatment may be from about 1 mg to about 10 mg, dosed daily. A dosing of a treatment may be from about 6 mg to about 7 mg, dosed daily. A dosing of a treatment may be dosed in more than one dose. A dosing of a treatment may be dosed daily. A dosing of a treatment may be dosed bi-weekly.

A dosing of a treatment may be from about 200 mg to about 400 mg, dosed daily. A dosing of a treatment may be from about 150 mg to about 450 mg, dosed daily.

A dosing of a treatment may be about 150 mg, dosed daily. A dosing of a treatment may be from about 100 mg to about 200 mg, dosed daily. A dosing of a treatment may be from about 125 mg to about 175 mg, dosed daily.

A dosing of a treatment may be about 200 mg, dosed daily. A dosing of a treatment may be from about 100 mg to about 200 mg, dosed daily. A dosing of a treatment may be from about 150 mg to about 250 mg, dosed daily.

A dosing of a treatment may be about 3.6 mg, dosed every 28 days. A dosing of a treatment may be from about 3 mg to about 4 mg, dosed every 28 days. A dosing of a treatment may be from about 2.5 mg to about 5.5 mg, dosed every 28 days.

A dosing of a treatment may be from about 5 mg to about 15 mg, dosed daily. A dosing of a treatment may be from about 1 mg to about 20 mg, dosed daily.

A dosing of a treatment may be from about 2.5 mg to about 10 mg, dosed daily. A dosing of a treatment may be from about 2 mg to about 12 mg, dosed daily. A dosing of a treatment may be from about 1 mg to about 15 mg, dosed daily.

Administration a treatment disclosed herein can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 120, 140, 160, 180, 200, 220, 240, 260, 280, 300, 320, 340, 360, 380, 400 days consecutive or nonconsecutive days. In some cases, a treatment duration can be from about 1 to about 30 days, from about 30 days to about 90 days, from about 60 days to about 210 days, from about 90 days to about 180 days, from about 90 days to about 360 days, or from about 180 days to about 360 days. In some cases, the treatment duration can be from about 90 days to about 180 days.

Administration of a treatment may be given to a subject one time, such as a one-time treatment. Administration of a treatment may be given to the subject two times or more, such as a two-time treatment, such as when a first-time treatment failed.

Administration or application of composition disclosed herein can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration or application of composition disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some cases, a composition can be administered or applied as a single dose or as divided doses. In some cases, the compositions described herein can be administered at a first time point and a second time point. In some cases, a composition can be administered such that a first administration is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

### Methods of Detection of Variants

In some cases, the disclosure provides methods to detect one or more genetic variants (e.g., in Table 1 or Table 2, or of a gene disclosed in FIG. 5). In some cases, the methods include selecting a panel of the one or more genetic variants for detection. In some cases, a genetic variant in a panel may comprise two or more genetic variants defining a minor allele. In some instances, the detecting may comprise: DNA sequencing, hybridizing with a complementary probe, performing an oligonucleotide ligation assay, performing a PCR-based assay, or any combination thereof. In some instances, the panel may comprise at least: 2, 3, 4, 5, 6, 7, 8, 9, 10, 15, 20, 25, 30, 35, 40, 45, 50, 60, 70, 75, 80, 90, 100, 150, 200, 250, 300, 350, 400, 450, 500, or more genetic variants defining minor alleles disclosed herein. In some instances, the genetic variant to detect or detected has an odds ratio (OR) of at least: 0.1, 1, 1.5, 2, 5, 10, 20, 50, 100, 127, 130, 140, 150, 200, 300, 400, 500, 600, 700, 800, 900, 1000, 1500, 2000, 2500, 3000, 3500, 4000, 4500, 5000, or more.

In some cases, a genetic variant may include single nucleotide polymorphisms (SNPs), insertion deletion polymorphisms (indels), damaging mutation variants, loss of function variants, synonymous mutation variants, nonsynonymous mutation variants, nonsense mutations, recessive markers, splicing/splice-site variants, frameshift mutation, insertions, deletions, genomic rearrangements, stop-gain, stop-loss, Rare Variants (RVs), translocations, inversions, and substitutions.

Genetic variants, for example SNPs, may be preceded and followed by highly conserved sequences that vary in less than 1/100 or 1/1000 members of the population. An individual may be homozygous or heterozygous for an allele at each SNP position. A SNP may, in some instances, be referred to as a “cSNP” to denote that the nucleotide sequence containing the SNP is an amino acid “coding” sequence. A SNP may arise from a substitution of one nucleotide for another at the polymorphic site. Substitutions can be transitions or transversions. A transition may be the replacement of one purine nucleotide by another purine nucleotide, or one pyrimidine by another pyrimidine. A transversion may be the replacement of a purine by a pyrimidine, or vice versa.

A synonymous codon change, or silent mutation is one that does not result in a change of amino acid due to the degeneracy of the genetic code. A substitution that changes a codon coding for one amino acid to a codon coding for a different amino acid (i.e., a non-synonymous codon change) is referred to as a missense mutation. A nonsense mutation may result in a type of non-synonymous codon change in which a stop codon is formed, thereby leading to premature termination of a polypeptide chain and a truncated protein. A read-through mutation is another type of non-synonymous codon change that causes the destruction of a stop codon, thereby resulting in an extended polypeptide product. An indel that occur in a coding DNA segment gives rise to a frameshift mutation.

Causative genetic variants may be those that produce alterations in gene expression or in the structure and/or function of a gene product, and therefore may be predictive of responsiveness to a pharmaceutical treatment, a possible clinical phenotype, or a combination thereof. One such class may include SNPs falling within regions of genes encoding a polypeptide product, i.e. cSNPs. These SNPs may result in an alteration of the amino acid sequence of the polypeptide product (i.e., non-synonymous codon changes) and may give rise to the expression of a defective or other variant protein. Furthermore, in the case of nonsense mutations, a SNP may lead to premature termination of a polypeptide product. Such variant products can result in a pathological condition, e.g., genetic endometriosis.

An association study of a genetic variant and a responsiveness to a treatment may involve determining the presence or frequency of the genetic variant in genetic material from subjects with the disorder of interest, such as endometriosis, and comparing the information to that of controls (i.e., individuals who respond or do not respond to the treatment, also referred to as “responders” or “nonresponders”) who are for example of similar age and race and received a similar dosage of the pharmaceutical composition. The appropriate selection of subjects and controls may be important to the success of genetic variant association studies. Therefore, a pool of individuals with well-characterized responses to the pharmaceutical composition and well-characterized genetic variant analysis may be extremely desirable.

A genetic variant may be screened in a tissue sample or any biological sample obtained from an affected individual, and compared to control samples, and selected for its increased (or decreased) occurrence in a specific condition, such as responsiveness or lack of response to a pharmaceutical composition, or to a pathology related to endometriosis, or a combination thereof. Once a statistically significant association is established between one or more variant(s) and a response to a pharmaceutical condition (or other phenotype) of interest, then the region around the genetic variant can optionally be thoroughly screened to identify the causative genetic locus/sequence(s) (e.g., causative variant/mutation, gene, regulatory region, etc.) that influences the condition or phenotype. Association studies may be conducted within the general population and are not limited to studies performed on related individuals in affected families (linkage studies). For diagnostic and prognostic purposes, if a particular genetic variant site is found to be useful for diagnosing a disease, such as endometriosis, or predicting a responsiveness to a particular pharmaceutical composition than other genetic variant sites which may be in linkage disequilibrium (LD) with this genetic variant site may also be expected to be useful for diagnosing the condition or predicting a responsiveness to a pharmaceutical composition. Linkage disequilibrium can be described in the human genome as blocks of genetics variants along a chromosome segment that do not segregate independently (i.e., that are non-randomly co-inherited). The starting (5′ end) and ending (3′ end) of these blocks can vary depending on the criteria used for linkage disequilibrium in a given database, such as the value of D′ or r² used to determine linkage disequilibrium.

In some instances, genetic variants can be identified in a study using a whole-genome case-control approach to identify single nucleotide polymorphisms that were closely associated with the development of endometriosis or predictive of responsive to a treatment, as well as genetic variants found to be in linkage disequilibrium with (i.e., within the same linkage disequilibrium block as) the endometriosis-associated variants, which can provide haplotypes (i.e., groups of variants that are co-inherited) to be readily inferred. Thus, the disclosure provides individual genetic variants associated with endometriosis or predictive of responsiveness to a treatment, as well as combinations of variants and haplotypes in genetic regions associated with endometriosis, methods of detecting these polymorphisms in a test sample, methods of determining the risk of an individual of having or developing endometriosis and for clinical sub-classification of endometriosis.

In some instances, one or more variant alleles of the disclosure can be associated with either an increased risk of having or developing endometriosis, a decreased risk of having or developing endometriosis, an increased probability of responding to a treatment, a decreased probability of responding to a treatment, or any combination thereof. Variant alleles that are associated with a decreased risk may be referred to as “protective” alleles, and variant alleles that are associated with an increased risk may be referred to as “susceptibility” alleles, “risk factors”, or “high-risk” alleles. Thus, whereas certain variants can be assayed to determine whether an individual possesses a variant allele that is indicative of an increased risk of having or developing endometriosis (i.e., a susceptibility allele), other variants can be assayed to determine whether an individual possesses a variant allele that is indicative of a decreased risk of having or developing endometriosis (i.e., a protective allele). Similarly, variant alleles of the disclosure can be associated with either an increased or decreased likelihood of responding to a treatment. The term “altered” may be used herein to encompass either of these two possibilities (e.g., an increased or a decreased risk/likelihood).

In some instances, nucleic acid molecules may be double-stranded molecules and that reference to a site on one strand refers, as well, to the corresponding site on a complementary strand. In defining a variant position, variant allele, or nucleotide sequence, reference to an adenine, a thymine (uridine), a cytosine, or a guanine at a site on one strand of a nucleic acid molecule also defines the complementary thymine (uridine), adenine, guanine, or cytosine (respectively) at the corresponding site on a complementary strand of the nucleic acid molecule. Thus, reference may be made to either strand in order to refer to a variant position, variant allele, or nucleotide sequence. Probes and primers may be designed to hybridize to either strand and variant genotyping methods disclosed herein may generally target either strand. Throughout the specification, in identifying a variant position, reference is generally made to the forward or “sense” strand, solely for the purpose of convenience. Since endogenous nucleic acid sequences exist in the form of a double helix (a duplex comprising two complementary nucleic acid strands), it is understood that the variants disclosed herein will have counterpart nucleic acid sequences and variants associated with the complementary “reverse” or “antisense” nucleic acid strand. Such complementary nucleic acid sequences, and the complementary variants present in those sequences, are also included within the scope of the disclosure.

Disclosed herein are methods for detecting genetic variants in a nucleic acid sample. The method can comprise sequencing a nucleic acid sample obtained from a subject. The subject may have endometriosis or be suspected of having endometriosis. The sequencing may comprise a high throughput method. The high throughput method can comprise nanopore sequencing. The method can comprise detecting one or more genetic variants in a nucleic acid sample, wherein the one or more genetic variants are listed in Table 1 or Table 2 or may be of genes listed in FIG. 5, or a combination thereof. The nucleic acid sample can comprise RNA. The RNA can comprise mRNA. The nucleic acid sample can comprise DNA. The DNA can comprise cDNA, genomic DNA, sheared DNA, cell free DNA, fragmented DNA, or PCR amplified products produced therefrom, or any combination thereof. The one or more genetic variants can comprise a genetic variant defining a minor allele. The one or more genetic variants can comprise at least about: 5, 10, 15, 20, 25, 50, 75, 100, 150, 200, 250, 500, or more genetic variants. The detection of the one or more genetic variants can have an odds ratio (OR) for responsiveness to a pharmaceutical composition of at least about: 1.5, 2, 5, 10, 20, 50, 100, or more. The one or more genetic variants can comprise a synonymous mutation, a non-synonymous mutation, a stop-gain mutation, a nonsense mutation, an insertion, a deletion, a splice-site variant, a frameshift mutation, or any combination thereof. The one or more genetic variants can comprise a protein damaging mutation. The genetic variant can comprise a variant in SPPL2C, MAP3K15, or any combinations thereof. The genetic variant can comprise a variant selected from the group consisting of SPPL2C, MAP3K15, or any combinations thereof. The method can comprise detecting one or more additional variants listed in Table 1 or Table 2. The one or more genetic variants can be identified based on a predictive computer algorithm. The one or more genetic variants can be identified based on reference to a database. The method can further comprise identifying a subject as a responder or non-responder to a treatment of endometriosis, as having endometriosis or being at risk of developing endometriosis, or any combination thereof. The method can comprise identifying a subject as a responder or a non-responder to a treatment of a disease or condition, or as having endometriosis or being at risk of developing endometriosis with a specificity of at least: 80%, 85%, 90%, 95%, 96%, 97%, 98%, or 99%. The method can comprise identifying a subject as a responder or a non-responder to a treatment of a disease or condition, or as having endometriosis or being at risk of developing endometriosis with a sensitivity of at least: 80%, 85%, 90%, 95%, 96%, 97%, 98%, or 99%. The method can comprise identifying a subject as a responder or a non-responder to a treatment of a disease or condition, or as having endometriosis or being at risk of developing endometriosis with an accuracy of at least: 80%, 85%, 90%, 95%, 96%, 97%, 98%, or 99%. The method can comprise identifying a subject as a responder or non-responder to a disease or condition. The method can comprise identifying a subject as having endometriosis. The subject can be asymptomatic for endometriosis. In some cases, the subject can have endometriosis and be asymptomatic. The subject can be symptomatic for endometriosis. The subject can be identified as a responder or a non-responder to the treatment. The subject can be identified as being at risk of developing endometriosis. The method can comprise selecting a treatment for the subject. The method can comprise administering a treatment to a subject. The treatment may comprise a pharmaceutical composition. The treatment may comprise an FDA-approved composition for the treatment or prevention of endometriosis. The treatment may comprise leuprolide acetate, a derivative thereof, a biosimilar thereof, an interchangeable thereof, or a salt thereof. The treatment can comprise administration of a hormonal therapy, an advanced reproductive technology therapy, a pain managing medication, or any combination thereof. The treatment can comprise administration of a hormonal contraceptive, a gonadotropin-releasing hormone (Gn-RH) receptor agonist, a gonadotropin-releasing hormone (Gn-RH) receptor antagonist, progestin, danazol, a biosimilar of any of these, an interchangeable of any of these, or any combination thereof. The therapeutic can comprise a pain medication. The pain medication can comprise a nonsteroidal anti-inflammatory drug (NSAID), ibuprofen, naproxen, an opioid, a cannabis-based therapeutic, or any combination thereof. The treatment may comprise an administration of a stem cell. In some cases, the one or more genetic variants may be listed in Table 1 or Table 2 or of a gene listed in FIG. 5. The method can further comprise identifying a subject as having endometriosis-associated infertility or being at risk of developing endometriosis-associated infertility. The method can further comprise administering assisted reproductive technology therapy to a subject. The assisted reproductive technology therapy can comprise in vitro fertilization, gamete intrafallopian transfer, or any combination thereof. The method can further comprise administering, intrauterine insemination or ovulation induction. A subject described herein can be a mammal. The mammal can be a human. Nanopore sequencing can be performed with a biological nanopore, a solid state nanopore, or a hybrid nanopore. Methods disclosed herein can detect 1, 5, 10, 15, 20, 30, 50, 60, 100, 80, 90, 100, 200 or more variants disclosed herein. Genetic variants detected herein can indicate endometriosis or a risk of developing endometriosis. In some embodiments, one or more genetic variant listed in Table 1 or Table 2 are the genetic variants detected.

### Genotyping Methods

In some cases, the process of determining which specific nucleotide (i.e., allele) is present at each of one or more variant positions, such as a variant position in a nucleic acid molecule characterized by a variant, is referred to as variant genotyping. The disclosure provides methods of variant genotyping, such as for use in screening for endometriosis or related pathologies, or determining predisposition thereto, or determining responsiveness to a form of treatment, or in genome mapping or variant association analysis, or any combination thereof.

Nucleic acid samples can be genotyped to determine which allele(s) is/are present at any given genetic region (e.g., variant position) of interest by methods well known in the art. The neighboring sequence can be used to design variant detection reagents such as oligonucleotide probes, which may optionally be implemented in a kit format. Common variant genotyping methods include, but are not limited to, TaqMan assays, molecular beacon assays, nucleic acid arrays, allele-specific primer extension, allele-specific PCR, arrayed primer extension, homogeneous primer extension assays, primer extension with detection by mass spectrometry, mass spectrometry with or with monoisotopic dNTPs (pyrosequencing, multiplex primer extension sorted on genetic arrays, ligation with rolling circle amplification, homogeneous ligation, OLA, multiplex ligation reaction sorted on genetic arrays, restriction-fragment length polymorphism, single base extension-tag assays, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, electrospray mass spectrometry, and electrical detection.

Various methods for detecting polymorphisms can include, but are not limited to, methods in which protection from cleavage agents is used to detect mismatched bases in RNA/RNA or RNA/DNA duplexes, comparison of the electrophoretic mobility of variant and wild type nucleic acid molecules, and assaying the movement of polymorphic or wild-type fragments in polyacrylamide gels containing a gradient of denaturant using denaturing gradient gel electrophoresis (DGGE). Sequence variations at specific locations can also be assessed by nuclease protection assays such as RNase and SI protection or chemical cleavage methods.

In some instances, a variant genotyping can be performed using the TaqMan assay, which is also known as the 5′ nuclease assay. The TaqMan assay may detect the accumulation of a specific amplified product during PCR. The TaqMan assay may utilize an oligonucleotide probe labeled with a fluorescent reporter dye and a quencher dye. The reporter dye may be excited by irradiation at an appropriate wavelength, it transfers energy to the quencher dye in the same probe via a process called fluorescence resonance energy transfer (FRET). When attached to the probe, the excited reporter dye may not emit a signal. The proximity of the quencher dye to the reporter dye in the intact probe may maintain a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter may be reduced. During PCR, the 5′ nuclease activity of DNA polymerase may cleave the probe, thereby separating the reporter dye and the quencher dye and resulting in increased fluorescence of the reporter. Accumulation of PCR product may be detected directly by monitoring the increase in fluorescence of the reporter dye. The DNA polymerase cleaves the probe between the reporter dye and the quencher dye only if the probe hybridizes to the target variant-containing template which is amplified during PCR, and the probe may be designed to hybridize to the target variant site only if a variant allele is present. TaqMan primer and probe sequences can readily be determined using the variant and associated nucleic acid sequence information provided herein. A number of computer programs, such as Primer Express (Applied Biosystems, Foster City, Calif.), can be used to rapidly obtain optimal primer/probe sets. It will be apparent to one of skill in the art that such primers and probes for detecting the variants of the disclosure are useful in diagnostic assays for endometriosis and related pathologies and can be readily incorporated into a kit format. The disclosure also includes modifications of the Taqman assay well known in the art such as the use of Molecular Beacon probes and other variant formats.

In some instances, a method for genotyping the variants can be the use of two oligonucleotide probes in an OLA. In this method, one probe may hybridize to a segment of a target nucleic acid with its 3′ most end aligned with the variant site. A second probe may hybridize to an adjacent segment of the target nucleic acid molecule directly 3′ to the first probe. The two juxtaposed probes may hybridize to the target nucleic acid molecule and may be ligated in the presence of a linking agent such as a ligase if there is perfect complementarity between the 3′ most nucleotide of the first probe with the variant site. If there is a mismatch, ligation may not occur. After the reaction, the ligated probes may be separated from the target nucleic acid molecule and detected as indicators of the presence of a variant.

In some instances, a method for variant genotyping may be based on mass spectrometry. Mass spectrometry takes advantage of the unique mass of each of the four nucleotides of DNA. variants can be unambiguously genotyped by mass spectrometry by measuring the differences in the mass of nucleic acids having alternative variant alleles. MALDI-TOF (Matrix Assisted Laser Desorption Ionization-Time of Flight) mass spectrometry technology is exemplary for extremely precise determinations of molecular mass, such as variants. Numerous approaches to variant analysis have been developed based on mass spectrometry. Exemplary mass spectrometry-based methods of variant genotyping include primer extension assays, which can also be utilized in combination with other approaches, such as traditional gel-based formats and microarrays.

In some instances, a method for genotyping the variants of the disclosure is the use of electrospray mass spectrometry for direct analysis of an amplified nucleic acid. In this method, in one aspect, an amplified nucleic acid product may be isotopically enriched in an isotope of oxygen (O), carbon (C), nitrogen (N) or any combination of those elements. In an exemplary embodiment the amplified nucleic acid may be isotopically enriched to a level of greater than 99.9% in the elements of O¹⁶, C¹² and N¹⁴. The amplified isotopically enriched product can then be analyzed by electrospray mass spectrometry to determine the nucleic acid composition and the corresponding variant genotyping. Isotopically enriched amplified products result in a corresponding increase in sensitivity and accuracy in the mass spectrum. In another aspect of this method an amplified nucleic acid that is not isotopically enriched can also have composition and variant genotype determined by electrospray mass spectrometry.

In some instances, variants can be scored by direct DNA sequencing. The nucleic acid sequences of the disclosure enable one of ordinary skill in the art to readily design sequencing primers for such automated sequencing procedures. Commercial instrumentation, such as the Applied Biosystems 377, 3100, 3700, 3730, and 3730.times.1 DNA Analyzers (Foster City, Calif.), is commonly used in the art for automated sequencing.

Variant genotyping can include the steps of, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target variant under conditions such that hybridization and amplification of the target nucleic acid region occurs, and determining the nucleotide present at the variant position of interest, or, in some assays, detecting the presence or absence of an amplification product (assays can be designed so that hybridization and/or amplification will only occur if a particular variant allele is present or absent). In some assays, the size of the amplification product is detected and compared to the length of a control sample; for example, deletions and insertions can be detected by a change in size of the amplified product compared to a normal genotype.

In some instances, a variant genotyping can be used in applications that include, but are not limited to, variant-endometriosis association analysis, endometriosis predisposition screening, endometriosis diagnosis, endometriosis prognosis, endometriosis progression monitoring, determining therapeutic strategies based on an individual's genotype, selecting a treatment, and stratifying a patient population for clinical trials for a treatment such as minimally invasive device for the treatment of endometriosis.

### Analysis of Rare and Private Mutations in Sequenced Endometriosis Genes

In some cases, the disclosure provides an analysis to evaluate a coding region of a gene as a component of a genetic diagnostic, a predictive test for endometriosis, or a predictive test to identify subjects that will respond to a treatment. In some instances, the analysis can comprise one or more of the approaches disclosed herein.

In some instances, the analysis can comprise performing DNA variant search on the next generation sequencing output file using a standard software designed for this purpose, for example Life Technologies TMAP algorithm with their default parameter settings, and Life Technologies Torrent Variant Caller software. ANNOVAR can be used to classify coding variants as synonymous, missense, frameshift, splicing, stop-gain, or stop-loss. Variants can be considered “loss-of-function” if the variant causes a stop-loss, stop-gain, splicing, or frame-shift insertion or deletion).

In some instances, the analysis can comprise evaluating prediction of an effect of each variant on protein function in silico using a variety of different software algorithms: Polyphen 2, Sift, Mutation Accessor, Mutation Taster, FATHMM, LRT, MetaLR, or any combination thereof. Missense variants can be deemed “damaging” if they are predicted to be damaging by at least one of the seven algorithms tested.

In some instances, the analysis can comprise searching population databases (e.g., gnomAD) and proprietary endometriosis allele frequency databases for the prevalence of any loss of function or damaging mutations identified by these analyses. The log of the odds ratio can be used to weight the marker when the variant has been previously observed in the reference databases. When a damaging variant or loss of function variant has never been reported in the reference databases, a default odds ratio of 10 can be used to weight the finding.

In some instances, the analysis can comprise incorporating findings into the Risk Score as with the other low-frequency alleles. Risk Score=Summation [log(OR)×Count], where count equals the number of low frequency alleles detected at each endometriosis associated locus. Risk scores can be converted to probability using a nomogram based on confirmed diagnoses.

In some instances, the methods of the disclosure can provide a high sensitivity of detecting gene mutations, diagnosing endometriosis, or identifying subjects that will respond to a treatment that is greater than 60%, 65%, 70%, 75%, 80%, 85%, 90%, 91%, 92%, 93%, 94%, 95%, 95.5%, 96%, 96.5%, 97%, 97.5%, 98%, 98.5%, 99%, 99.5% or more. In some instances, the methods disclosed herein can provide a high specificity of detecting and classifying gene mutations, diagnosing endometriosis, or identifying subjects that will respond to a treatment, for example, greater than 80%, 85%, 90%, 91%, 92%, 93%, 94%, 95%, 95.5%, 96%, 96.5%, 97%, 97.5%, 98%, 98.5%, 99%, 99.5% or more. In some instances, a nominal specificity for the method disclosed herein can be greater than or equal to 70%. In some instances, a nominal Negative Predictive Value (NPV) for the method disclosed herein can be greater than or equal to 95%. In some instances, an NPV for the method disclosed herein can be about 95%, 95.5%, 96%, 96.5%, 97%, 97.5%, 98%, 98.5%, 99%, 99.5% or more. In some instances, a nominal Positive Predictive Value (PPV) for the method disclosed herein can be greater than or equal to 95%. In some instances, a PPV for the method disclosed herein can be about 95%, 95.5%, 96%, 96.5%, 97%, 97.5%, 98%, 98.5%, 99%, 99.5% or more. In some instances, the accuracy of the methods disclosed herein in diagnosing endometriosis or identifying subjects that will response to a treatment can be greater than 70%, 75%, 80%, 85%, 90%, 91%, 92%, 93%, 94%, 95%, 95.5%, 96%, 96.5%, 97%, 97.5%, 98%, 98.5%, 99%, 99.5% or more.

### Computer Implemented Methods

In some cases, the disclosure provides methods for analysis of gene sequence data associated software and computer systems. The method, for example being computer implemented, can enable a clinical geneticist or other healthcare technician to sift through vast amounts of gene sequence data, to identify potential disease-causing genomic variants or to identify subjects that may respond or not respond to a treatment. In some cases, the gene sequence data is from a patient who may be suspected of having a genetic disorder such as endometriosis.

In some cases, provided herein is a method for identifying a subject as a responder or non-responder to a treatment for a disease or condition, such as endometriosis. In some cases, provided herein is a method identifying a genetic disorder such as endometriosis or predicting a risk thereof in an individual, or identifying a genetic variant that is causative of a phenotype in an individual. In some instances, the method can comprise determining gene sequence for a patient suspected of having a genetic disorder, identifying sequence variants, annotating the identified variants based on one or more criteria, and filtering or searching the variants at least partially based on the annotations, to thereby identify the subject as a responder or non-responder to the treatment, identify the potential disease-causing variants, or a combination thereof.

In some instances, the gene sequence is obtained by use of a sequencing instrument, or alternatively, gene sequence data is obtained from another source, such as for example, a commercial sequencing service provider. Gene sequence can be chromosomal sequence, cDNA sequence, or any nucleotide sequence information that allows for detection of responsiveness to a treatment or for detection of a genetic disease. Generally, the amount of sequence information is such that computational tools may be required for data analysis. For example, the sequence data may represent at least half of the individual's genomic or cDNA sequence (e.g., of a representative cell population or tissue), or the individuals entire genomic or cDNA sequence. In various embodiments, the sequence data comprises the nucleotide sequence for at least 1 million base pairs, at least 10 million base pairs, or at least 50 million base pairs. In certain embodiments, the DNA sequence is the individual's exome sequence or full exonic sequence component (i.e., the exome; sequence for each of the exons in each of the known genes in the entire genome). In some embodiments, the source of genomic DNA or cDNA may be any suitable source and may be a sample particularly indicative of a disease or phenotype of interest. In certain embodiments, the source of the sample is a tissue or sample that is potentially malignant.

In some instances, the gene sequence may be mapped with one or more reference sequences to identify sequence variants. For example, the base reads are mapped against a reference sequence, which in various embodiments is presumed to be a “normal” non-disease sequence. The DNS sequence derived from the Human Genome Project is generally used as a “premier” reference sequence. A number of mapping applications are known, and include TMAP, BWA, GSMAPPER, ELAND, MOSAIK, and MAQ. Various other alignment tools are known and could also be implemented to map the base reads.

In some cases, based on the sequence alignments, and mapping results, sequence variants can be identified. Types of variants may include insertions, deletions, indels (a colocalized insertion and deletion), damaging mutation variants, loss of function variants, synonymous mutation variants, nonsynonymous mutation variants, nonsense mutations, recessive markers, splicing/splice-site variants, frameshift mutation, insertions, deletions, genomic rearrangements, stop-gain, stop-loss, Rare Variants (RVs), translocations, inversions, and substitutions. While the type of variants analyzed is not limited, the most numerous of the variant types will be single nucleotide substitutions, for which a wealth of data is currently available. In various embodiments, comparison of the test sequence with the reference sequence will produce at least 500 variants, at least 1000 variants, at least 3,000 variants, at least 5,000 variants, at least 10,000 variants, at least 20,000 variants, or at least 50,000 variants, but in some embodiments, will produce at least 1 million variants, at least 2 million variants, at least 3 million variants, at least 4 million variants, or at least 10 million variants. The tools provided herein enable the user to navigate the vast amounts of genetic data to identify potentially disease-causing variants.

In some cases, a wealth of data can be extracted for the identified variants, including one or more of conservation scores, genic/genomic location, zygosity, SNP ID, Polyphen, FATHMM, LRT, Mutation Accessor, and SIFT predictions, splice site predictions, amino acid properties, disease associations, annotations for known variants, variant or allele frequency data, and gene annotations. Data may be calculated and/or extracted from one or more internal or external databases. Since certain categories of annotations (e.g., amino acid properties/PolyPhen and SIFT data) are dependent on a nature of the region of the genome in which they are contained (e.g., whether a variant is contained within a region translated to give rise to an amino acid sequence in a resultant protein), these annotations can be carried out for each known transcript. Exemplary external databases include OMIM (Online Mendelian Inheritance in Man), HGMD (The Human Gene Mutation Database), PubMed, PolyPhen, SIFT, SpliceSite, reference genome databases, the University of California Santa Cruz (UCSC) genome database, CLINVAR database, the BioBase biological databases, the dbSNP Short Genetic Variations database, the Rat Genome Database (RGD), and/or the like. Various other databases may be employed for extracting data on identified variants. Variant information may be further stored in a central data repository, and the data extracted for future sequence analyses.

In some instances, variants may be tagged by the user with additional descriptive information to aid subsequent analysis. For example, confidence in the existence of the variant can be recorded as confirmed, preliminary, or sequence artifact. Certain sequencing technologies tend to produce certain types of sequence artifacts, and the method herein can allow such suspected artifacts to be recorded. The variants may be further tagged in basic categories of benign, pathogenic, or unknown, or as potentially of interest.

In some instances, queries can be run to identify variants meeting certain criteria, or variant report pages can be browsed by chromosomal position or by gene, the latter allowing researchers to focus on only those variations that exist in a particular set of genes of interest. In some embodiments, the user selects only variants with well-documented and published disease associations (e.g., by filtering based on HGMD or other disease annotation). Alternatively, the user can filter for variants not previously associated with disease, but of a type likely to be deleterious, such as those introducing frameshifts, non-synonymous substitutions (predicted by Polyphen or SIFT), or premature terminations. Further, the user can exclude from analysis those variants believed to be neutral (based on their frequency of occurrence in studies populations), for example, through exclusion of variants in dbSNP. Additional exclusion criteria include mode of inheritance (e.g., heterozygosity), depth of coverage, and quality score.

In certain embodiments, base calling is carried out to extract the sequence of the sequencing reads from an image file produced by an instrument scanner. Following base calling and base quality trimming/filtering, the reads are mapped against a reference sequence (assumed to be normal for the phenotype under analysis) to identify variations (variants) between the two with the assumption that one or more of these differences will be associated with phenotype of the individual whose DNA is under analysis. Subsequently, each variant is annotated with data that can be used to determine the likelihood that that variant is associated with the phenotype under analysis. The analysis may be fully or partially automated as described in detail below and may include use of a central repository for data storage and analysis, and to present the data to analysts and clinical geneticists in a format that makes identification of variants with a high likelihood of being associated with the phenotypic difference more efficient and effective.

In some embodiments, a user can be provided with the ability to run cross sample queries where the variants from multiple samples are interrogated simultaneously. In such embodiments, for example, a user can build a query to return data on only those variants that are exactly shared across a user defined group of samples. This can be useful for family-based analyses where the same variant is believed to be associated with disease in each of the affected family members. For another example, the user can also build a query to return only those variants that are present in genes where the gene contains at least one, but not necessarily the same, variant. This can be useful where a group of individuals with disease are not related (the variants associated with the disease are not necessary exactly the same, but result in a common alteration in normal function). For yet another example, the user can specify to ignore genes containing variants in a user defined group of samples. This can be useful to exclude polymorphisms (variants believed or confirmed not to be associated with disease) where the user has access to a user defined group of control individuals who are believed to not have the disease associated variant. For each of these queries a user can additionally filter the variants by specifying any or all the previously discussed filters on top of the cross-sample analyses. This allows a user to identify variants matching these criteria, which are shared between or segregated amongst samples.

For example, a variant analysis system can be implemented locally, or implemented using a host device and a network or cloud computing. For example, the variant analysis system can be software stored in memory of a personal computing device (PC) and implemented by a processor of the PC. In such embodiments, for example, the PC can download the software from a host device and/or install the software using any suitable device such as a compact disc (CD).

The method may employ a computer-readable medium, or non-transitory processor-readable medium. Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

Examples of computer code can include, but are not limited to, micro-code or micro-instructions, machine instructions, such as produced by a compiler, code used to produce a web service, and files containing higher-level instructions that are executed by a computer using an interpreter. For example, embodiments may be implemented using Python, Java, C++, or other programming languages (e.g., object-oriented programming languages) and development tools. Additional examples of computer code can include, but are not limited to, control signals, encrypted code, and compressed code.

In some cases, variants provided herein may be “provided” in a variety of mediums to facilitate use thereof. As used in this section, “provided” can refer to a manufacture, other than an isolated nucleic acid molecule, that contains variant information of the disclosure. Such a manufacture provides the variant information in a form that allows a skilled artisan to examine the manufacture using means not directly applicable to examining the variants or a subset thereof as they exist in nature or in purified form. The variant information that may be provided in such a form includes any of the variant information provided by the disclosure such as, for example, polymorphic nucleic acid and/or amino acid sequence information, information about observed variant alleles, alternative codons, populations, allele frequencies, variant types, and/or affected proteins, or any other information provided herein.

In some instances, the variants can be recorded on a computer readable medium. As used herein, “computer readable medium” can refer to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon a nucleotide sequence of the disclosure. One such medium is provided with the present application, namely, the present application contains computer readable medium (CD-R) that has nucleic acid sequences (and encoded protein sequences) containing variants provided/recorded thereon in ASCII text format in a Sequence Listing along with accompanying Tables that contain detailed variant and sequence information.

As used herein, “recorded” can refer to a process for storing information on computer readable medium. A skilled artisan can readily adopt any of the presently known methods for recording information on computer readable medium to generate manufactures comprising the variant information of the disclosure. A variety of data storage structures are available to a skilled artisan for creating a computer readable medium having recorded thereon a nucleotide or amino acid sequence of the disclosure. The choice of the data storage structure will generally be based on the means chosen to access the stored information. In addition, a variety of data processor programs and formats can be used to store the nucleotide/amino acid sequence information of the disclosure on computer readable medium. For example, the sequence information can be represented in a word processing text file, formatted in commercially-available software such as WordPerfect and Microsoft Word, represented in the form of an ASCII file, or stored in a database application, such as OB2, Sybase, Oracle, or the like. A skilled artisan can readily adapt any number of data processor structuring formats (e.g., text file or database) in order to obtain computer readable medium having recorded thereon the variant information of the disclosure.

By providing the variants in computer readable form, a skilled artisan can access the variant information for a variety of purposes. Computer software is publicly available which allows a skilled artisan to access sequence information provided in a computer readable medium. Examples of publicly available computer software include BLAST and BLAZE search algorithms.

In some cases, the disclosure can provide systems, particularly computer-based systems, which contain the variant information described herein. Such systems may be designed to store and/or analyze information on, for example, a large number of variant positions, or information on variant genotypes from a large number of individuals. The variant information of the disclosure represents a valuable information source. The variant information of the disclosure stored/analyzed in a computer-based system may be used for such computer-intensive applications as determining or analyzing variant allele frequencies in a population, mapping endometriosis genes, genotype-phenotype association studies, grouping variants into haplotypes, correlating variant haplotypes with response to particular treatments or for various other bioinformatic, pharmacogenomic or drug development.

As used herein, “a computer-based system” can refer to the hardware means, software means, and data storage means used to analyze the variant information of the disclosure. The minimum hardware means of the computer-based systems of the disclosure typically comprises a central processing unit (CPU), input means, output means, and data storage means. A skilled artisan can readily appreciate that any one of the currently available computer-based systems are suitable for use in the disclosure. Such a system can be changed into a system of the disclosure by utilizing the variant information provided on the CD-R, or a subset thereof, without any experimentation.

As stated above, the computer-based systems can comprise a data storage means having stored therein variants of the disclosure and the necessary hardware means and software means for supporting and implementing a search means. As used herein, “data storage means” can refer to memory which can store variant information of the disclosure, or a memory access means which can access manufactures having recorded thereon the variant information of the disclosure.

As used herein, “search means” can refer to one or more programs or algorithms that are implemented on the computer-based system to identify or analyze variants in a target sequence based on the variant information stored within the data storage means. Search means can be used to determine which nucleotide is present at a particular variant position in the target sequence. As used herein, a “target sequence” can be any DNA sequence containing the variant position(s) to be searched or queried.

A variety of structural formats for the input and output means can be used to input and output the information in the computer-based systems of the disclosure. An exemplary format for an output means is a display that depicts the presence or absence of specified nucleotides (alleles) at particular variant positions of interest. Such presentation can provide a rapid, binary scoring system for many variants simultaneously.

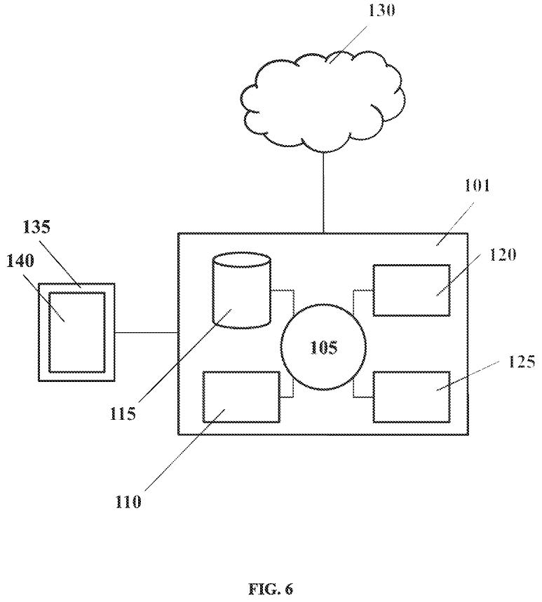

The disclosure provides computer control systems that are programmed to implement methods of the disclosure. FIG. 6 shows a computer system 101 that is programmed or otherwise configured for selection of an effective treatment of endometriosis, configured for endometriosis diagnosis, or a combination thereof. The computer system 101 can regulate various aspects of detection of genetic variants associated with endometriosis or indicative of a therapeutic effective of a particular treatment of the disclosure. The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine-readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example, for example a monitor. Examples of UI's include, without limitation, a graphical user interface (GUI) and web-based user interface.

Methods and systems of the disclosure can be implemented by way of one or more algorithms. An algorithm can be implemented by way of software upon execution by the central processing unit 105. The algorithm can, for example, Polyphen 2, Sift, Mutation Accessor, Mutation Taster, FATHMM, LRT, MetaLR, or any combination thereof.

### Specific Embodiments

A number of methods and systems are disclosed herein. Specific exemplary embodiments of these methods and systems are disclosed below.

Embodiment 1. A method comprising: (a) detecting a presence of at least one genetic variant of Table 1 in a genetic material obtained from a subject, wherein the subject has endometriosis or is at risk of developing endometriosis; and (b) treating the subject for the endometriosis with a therapeutically effective amount of a treatment that comprises leuprolide acetate, a derivative thereof, a biosimilar thereof, or an interchangeable thereof.

Embodiment 2. A method comprising: (a) detecting a presence of at least one genetic variant of Table 2 in a genetic material obtained from a subject, wherein the subject has endometriosis or is at risk of developing endometriosis; and (b) treating the subject for the endometriosis with a therapeutically effective amount of a treatment that does not comprise leuprolide acetate.

Embodiment 3. A method comprising: detecting a presence of at least one genetic variant of Table 1 in a genetic material obtained from a subject, wherein the subject has endometriosis or is at risk of developing endometriosis, wherein the presence of the at least one genetic variant of Table 1 is indicative of a therapeutically effective response to a treatment for treating the endometriosis, and wherein the treatment comprises leuprolide acetate, a derivative thereof, a biosimilar thereof, or an interchangeable thereof.

Embodiment 4. A method comprising: detecting a presence of at least one genetic variant of Table 2 in a genetic material obtained from a subject, wherein the subject has endometriosis or is a risk of developing endometriosis, wherein the presence of the at least one genetic variant of Table 2 is indicative of a therapeutically effective response to a treatment for treating the endometriosis, wherein the treatment does not comprise leuprolide acetate.

Embodiment 5. The method of embodiment 3 or embodiment 4, further comprising: treating the subject for the endometriosis.

Embodiment 6. The method of any one of embodiments 1-5, wherein the treating comprises prophylactic treating.

Embodiment 7. The method of any one of embodiments 1-6, further comprising: altering or updating the treatment based at least in part on the detecting.

Embodiment 8. The method of any one of embodiments 1-7, wherein the detecting occurs prior to administering the treatment to the subject.

Embodiment 9. The method of any one of embodiments 1-8, further comprising: selecting the treatment from a plurality of treatments.

Embodiment 10. The method of any one of embodiments 1-9, further comprising: obtaining the genetic material from the subject.

Embodiment 11. The method of any one of embodiments 1-10, further comprising: providing a recommendation to prescribe the treatment to the subject.

Embodiment 12. The method of any one of embodiments 1-11, wherein the subject has the endometriosis.

Embodiment 13. The method of any one of embodiments 1-11, wherein the subject is at risk of developing the endometriosis.

Embodiment 14. The method of any one of embodiments 1-13, wherein the subject suffers from pelvic pain.

Embodiment 15. The method of any one of embodiments 1-14, wherein the subject suffers from infertility.

Embodiment 16. The method of any one of embodiments 1-15, wherein the genetic material is obtained from a reproductive tissue, a blood sample, or a combination thereof.

Embodiment 17. The method of embodiment 16, wherein the genetic material is obtained from the reproductive tissue that comprises endometrial tissue, uterine tissue, ovarian tissue, fallopian tissue, cervical tissue, vulvar tissue, or any combination thereof.

Embodiment 18. The method of embodiment 17, wherein the genetic material is obtained from the reproductive tissue that comprises the endometrial tissue.

Embodiment 19. The method of embodiment 16, wherein the genetical material is obtained from the blood sample.

Embodiment 20. The method of any one of embodiments 1-19, wherein the genetic material comprises cell-free DNA.

Embodiment 21. The method of any one of embodiments 1-20, wherein the genetic material comprises RNA.

Embodiment 22. The method of any one of embodiments 1-21, wherein the genetic variant comprises at least two genetic variants.

Embodiment 23. The method of embodiment 1 or embodiment 3, wherein the genetic variant is of MAP3K15.

Embodiment 24. The method of any one of embodiments 1-21, wherein the genetic variant is of C17orf53, MTL5, SYT15, BCO2, ADD1, C14orf79, or any combination thereof.

Embodiment 25. The method of any one of embodiments 1-24, wherein the detecting comprises sequencing at least a portion of the genetic material.

Embodiment 26. The method of any one of embodiments 1-24, wherein the detecting comprises hybridizing a probe to a portion of the genetic material, wherein the probe is specific for the genetic variant.

Embodiment 27. The method of any one of embodiments 1-26, further comprising: measuring a total variant burden in at least a portion of the genetic material.

Embodiment 28. The method of any one of embodiments 1-27, further comprising: measuring a mood of the subject.

Embodiment 29. The method of any one of embodiments 1-28, further comprising: measuring a hormone receptor level in the genetic material.

Embodiment 30. The method of embodiment 29, wherein the hormone receptor level is an estrogen receptor level, a progesterone receptor level, or a combination thereof.

Embodiment 31. The method of embodiment 30, wherein the hormone receptor level is the estrogen receptor level.

Embodiment 32. The method of embodiment 30, wherein the hormone receptor level is the progesterone receptor level.

Embodiment 33. The method of any one of embodiments 1-32, wherein the treatment comprises administration of a gonadotropin releasing hormone (GnRH) or a synthetic analog thereof to the subject.

Embodiment 34. The method of any one of embodiments 1-32, wherein the treatment comprises administration of a GnRH receptor agonist, a GnRH receptor antagonist, a progestin, norethindrone, medroxyprogesterone, a biosimilar of any of these, an interchangeable of any of these, a salt of any of these, or any combination thereof.

Embodiment 35. The method of any one of embodiments 1-32, wherein the treatment comprises administration of RU-486 (CAS #84371-65-3), ethylnorgestrienone (CAS #16320-04-0), 2,3-isoxazolethisterone (CAS #17230-88-5), elagolix (CAS #834153-87-6), goserelin (CAS #65807-02-5), norethindrone acetate (CAS #38673-38-0), methylhydroxyprogesterone acetate (CAS #71-58-9), a biosimilar of any of these, an interchangeable of any of these, a salt of any of these, or any combination thereof.

Embodiment 36. The method of any one of embodiments 1-32, wherein the treatment comprises administration of a pharmaceutical composition in unit dose form.

Embodiment 37. The method of any one of embodiments 1-36, wherein the treatment comprises administration of a stem cell.

Embodiment 38. The method of any one of embodiments 1-37, wherein the treatment comprises administration of composition comprising: a cannabis, a nonsteroidal anti-inflammatory drug (NSAID), a progestin, a progesterone, or any combination thereof.

Embodiment 39. The method of embodiment 38, wherein the composition comprises the cannabis, the NSAID, and the progestin.

Embodiment 40. The method of embodiment 38, wherein the composition comprises the cannabis, the NSAID, and the progesterone.

Embodiment 41. The method of any one of embodiments 38-40, wherein the NSAID comprises ibuprofen, naproxen, or a combination thereof.

Embodiment 42. The method of any one of embodiments 36-41, wherein the composition further comprises human serum albumin.

Embodiment 43. The method of any one of embodiments 1-42, further comprising: comparing a result of the method to a reference.

Embodiment 44. The method of embodiment 43, wherein the reference comprises a derivative of the reference.

Embodiment 45. The method of embodiment 43, wherein the reference comprises a result of the method performed on a reference sample.

Embodiment 46. The method of embodiment 45, wherein the reference sample is of a subject responsive to the treatment.

Embodiment 47. The method of embodiment 43, wherein the comparing is performed by a computer processor.

Embodiment 48. The method of embodiment 43, wherein the comparing is performed by a trained algorithm.

Embodiment 49. The method of embodiment 43, wherein the reference comprises a result obtained from genetic material of a subject diagnosed with endometriosis.

Embodiment 50. The method of embodiment 43, wherein the reference comprises a result obtained from genetic material of a subject responsive to the treatment.

Embodiment 51. The method of any one of embodiments 1-50, further comprising: detecting an epigenetic marker in at least a portion of the genetic material.

Embodiment 52. The method of embodiment 51, wherein the epigenetic marker comprises a methylated marker, a hydroxymethylated marker, a carboxylated marker, a formylated marker, or any combination thereof.

Embodiment 53. The method of embodiment 51, wherein the portion comprising the epigenetic marker is RNA or DNA.

Embodiment 54. The method of any one of embodiments 1-53, further comprising: reporting a result of the method.

Embodiment 55. The method of embodiment 54, wherein the result comprises an output of the detecting.

Embodiment 56. The method of embodiment 54, wherein the reporting comprises electronic reporting.

Embodiment 57. The method of embodiment 1, further comprising: identifying the subject as a responder to the leuprolide acetate, the derivative thereof, the biosimilar thereof, or the interchangeable thereof.

Embodiment 58. The method of embodiment 2, further comprising: identifying the subject as a non-responder to the leuprolide acetate.

Embodiment 59. The method of embodiment 57 or embodiment 58, wherein the identifying is based in part on: a disease activity score; a presence, an absence, or a recurrence of pelvic pain; a cessation of the treatment; a scoring of dysmenorrhea; a presence of dyspareunia; a failure to conceive; a recurrence of a symptom following a treatment; a surgical intervention; or any combination thereof.

Embodiment 60. The method of embodiment 59, wherein the identifying is based on the presence, the absence, or the recurrence of pelvic pain.

Embodiment 61. The method of embodiment 60, wherein the presence, the absence or the recurrence of pelvic pain is reported by the subject on a visual analog scale (VAS).

Embodiment 62. The method of embodiment 60, wherein the presence, the absence or the recurrence of pelvic pain is reported after the treatment is completed.

Embodiment 63. The method of embodiment 60, wherein the pelvic pain comprises non-menstrual pelvic pain.

Embodiment 64. The method of embodiment 59, wherein the identifying is based on the disease activity score.

Embodiment 65. The method of embodiment 57 or embodiment 60, wherein the identifying is based at least in part on a medical history of the subject, a hormone receptor level of the subject, a mood of the subject, or any combination thereof.

Embodiment 66. The method of embodiment 57, wherein the subject is identified as the responder with a sensitivity of at least about 80%.

Embodiment 67. The method of embodiment 57, wherein the subject is identified as the responder with a specificity of at least about 80%.

Embodiment 68. The method of embodiment 60, wherein the subject is identified as the non-responder with a sensitivity of at least about 80%.

Embodiment 69. The method of embodiment 60, wherein the subject is identified as the non-responder with a specificity of at least about 80%.

### EXAMPLES

### Example 1

Objective: Inherited genetic differences in drug metabolic pathways can affect an individual patient's response to drugs (both therapeutic effects and adverse effects). Depot leuprolide acetate (LA) is used to treat endometriosis symptoms. A significant number of patients have little or no improvement with LA therapy, and metabolism of the drug is likely to be affected by several polymorphisms in cytochrome P450 genes, but to date there are no published pharmacogenetics studies regarding LA in the literature. We have discovered endometriosis associated genetic markers. For this study, we tested whether a patient's genetic “score” correlates with response to LA therapy.

Design: Retrospective cohort study

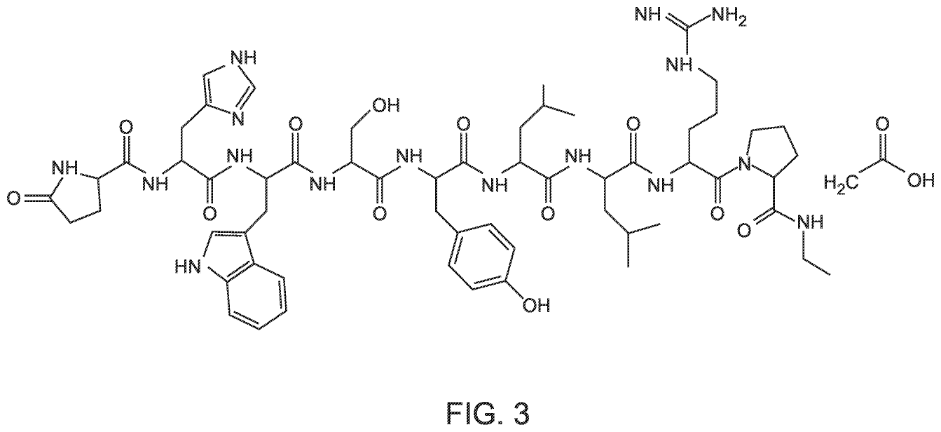

Materials and Methods: Caucasian women presenting with pelvic pain that were surgically diagnosed with endometriosis and treated with LA were included in this study. Referring to FIG. 3, leuprolide acetate (LA) is an FDA approved treatment for endometriosis. In a subset of women, little or no improvement with LA treatment is achieved.

Subjects were divided into two groups based on self-report of therapeutic effectiveness that were confirmed using medical records: 158 subjects reported significant symptomatic relief with LA therapy and 177 subjects reported no benefit from LA. Patients with minimal or uncertain benefit were excluded, DNA samples were tested for low-frequency variants associated with endometriosis and a genetic risk score was calculated. Genotype results for each marker was weighted using the log of the lower bound of 95% confidence interval of the observed odds ratio for endometriosis (as calculated by a discovery set of 2,360 endometriosis patients compared with 55,860 published gnomAD Non-Finnish European population controls). The comparison of the genetic score for the two study groups was performed using one-sided T test.

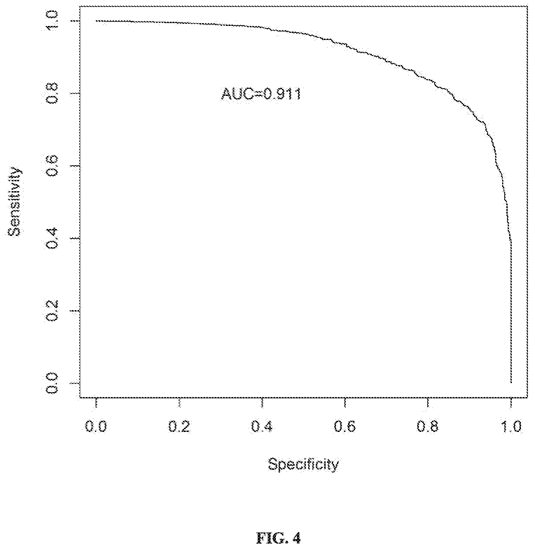

Referring to FIG. 4, 200 ancestry markers, 77 GWAS markers, and 712 low frequency mutations tested. Nine genes sequenced. An algorithm was employed to weight markers and to combine results into risk statement. DNA samples obtained from the subjects were tested for variants associated with endometriosis. Genotype results for each marker were weighted using the log of the lower bound of 95% confidence interval of the observed odds ratio. A genetic risk score was calculated for each subject. Genetic scores for study cohorts were compared using one-sided T test. Population controls had a mean endometriosis genetic risk score of 0.8. Subjects who reported significant symptomatic relief with LA therapy had a mean score equal to 8.4. Subjects who reported no benefit had a mean score of 6.8 (p=0.05).

Variants with Significant Differences: Referring to FIG. 5, variants with significant differences (all having p<0.005 comparing LA responders vs. non-responders) include C17orf53, MTL5, SYT15, BCO2, ADD1, and C14orf79. C17orf53 and MTL5 were variants predictive of leuprolide acetate success. SYT15, BCO2, ADD1, C14orf79 were variants predictive of leuprolide acetate failure.

Cytochrome P450 Genes: CYP genes control metabolism of over 85% of prescription drugs and affect activities of many other genes. Action of LA may be affected by polymorphisms in one or more cytochrome P450 genes. Small differences were observed: CYP2D6-non-functional “*4 allele” present in 19.7% of the population is seen in 13.6% of LA non-responders (p=0.003). CYP2D6 rapid metabolizer variant (rs16947) increased slightly in LA responders (p=0.05). CYP2B6 allele (24% in population controls) is present in 29% of LA non-responders (p=0.02).

Subjects who reported significant symptomatic relief with LA therapy had a mean genetic score (8.4) that was higher than those who reported no benefit (6.8) (p=0.05). Both responders and non-responders have higher genetic scores than population controls (0.8).

Conclusions: Disease associated DNA variants are present in almost every endometriosis patient studied, and these variants are likely to contribute to the clinical heterogeneity of endometriosis. Women who responded to LA therapy were likely to carry a higher burden of gene variants than non-responders. Subset of DNA markers may have greatest effect.

### Example 2

Caucasian women presenting with pelvic pain that were surgically diagnosed with endometriosis and treated with Leuprolide Acetate (LA) were included in this study. LA is a treatment for endometriosis. However, a subset of women receives little or no improvement with LA therapy. Subjects were divided into two groups based on self-report of therapeutic effectiveness: 163 reported significant symptomatic relief with LA therapy (LA responders) and 230 reported no benefit (LA non-responders). Patients with minimal or uncertain benefit were excluded.

DNA samples were tested for 3,287 low-frequency variants associated with endometriosis and a Genetic Risk Score (GRS) was calculated. GRS was calculated using genotype results for each marker weighted using the log of the lower bound of 95% confidence interval of the observed odds ratio in endometriosis discovery study. The comparison of the genetic score for the two study groups was performed using one-sided T test. We looked for association in LA responders vs LA non-responders among these 3,287 low frequency variants associated with endometriosis using Fisher's exact test. A significance threshold (alpha) of 0.2, minor allele frequency of 0.01 and a minimum odds ratio of 3 has at least 80% power to detect an associated variant with 163 LA responders and 230 LA non-responders. Table 1 shows the list of genetic variants (1-18) indicative of responders to LA therapy and Table 2 shows a list of genetic variants (19-48) indicative of non-responders to LA therapy.

TABLE 1

| | | | | REF | ALT | | | | | |
|---|---|---|---|---|---|---|---|---|---|---|
| | | Chromo- | | Allele | Allele | LupSuccess- | LupFailure- | | | |
| # | Damaginghits | some | Position | (0) | (1) | Freq | Freq | EndoFreq | gnomNFEfreq | p |
| 1 | 4 | chrX | 19379640 | G | C | 0.0000 | 0.0130 | 0.0093 | 0.0063 | 4.46E−02 |
| 2 | 5 | chr2 | 234229468 | C | T | 0.0000 | 0.0109 | 0.0053 | 0.0033 | 8.01E−02 |
| 3 | 0 | chr4 | 1805502 | C | T | 0.0000 | 0.0109 | 0.0066 | 0.0039 | 8.01E−02 |
| 4 | 0 | chr5 | 133328003 | A | G | 0.0000 | 0.0109 | 0.0070 | 0.0045 | 8.01E−02 |
| 5 | 0 | chr9 | 135946390 | G | C | 0.0000 | 0.0109 | 0.0040 | 0.0023 | 8.01E−02 |
| 6 | 1 | chr18 | 43311054 | G | A | 0.0000 | 0.0109 | 0.0038 | 0.0020 | 8.01E−02 |
| 7 | 0 | chr11 | 77378448 | C | T | 0.0031 | 0.0196 | 0.0076 | 0.0052 | 5.25E−02 |
| 8 | 5 | chr8 | 25174610 | C | T | 0.0031 | 0.0174 | 0.0108 | 0.0079 | 8.86E−02 |
| 9 | 1 | chr14 | 103396060 | A | G | 0.0031 | 0.0174 | 0.0087 | 0.0060 | 8.86E−02 |
| 10 | 6 | chr19 | 3546264 | C | T | 0.0031 | 0.0174 | 0.0095 | 0.0069 | 8.86E−02 |
| 11 | 0 | chr20 | 34092213 | G | A | 0.0031 | 0.0174 | 0.0117 | 0.0082 | 8.86E−02 |
| 12 | 1 | chr2 | 242207024 | T | A | 0.0031 | 0.0152 | 0.0091 | 0.0063 | 1.49E−01 |
| 13 | 1 | chr2 | 242312655 | C | T | 0.0031 | 0.0152 | 0.0089 | 0.0061 | 1.49E−01 |
| 14 | 0 | chr10 | 47087299 | G | C | 0.0031 | 0.0152 | 0.0068 | 0.0035 | 1.49E−01 |
| 15 | 4 | chr11 | 47469631 | G | T | 0.0031 | 0.0152 | 0.0047 | 0.0026 | 1.49E−01 |
| 16 | 0 | chr17 | 55917291 | G | A | 0.0031 | 0.0152 | 0.0110 | 0.0076 | 1.49E−01 |
| 17 | 0 | chr20 | 34078517 | G | A | 0.0031 | 0.0152 | 0.0112 | 0.0079 | 1.49E−01 |
| 18 | 5 | chr1 | 205901026 | C | T | 0.0061 | 0.0196 | 0.0125 | 0.0093 | 1.35E−01 |
| | | | | | | | | | | gnomad- |
| | | | | | | | | | | population- |
| # | OR | L95 | U95 | gene | type | nuclechange | aachange | transcript | dbsnp | Freq |
| 1 | 0.00 | 0.00 | NaN | MAP3K15 | nonsynonymous SNV | c.C3751G | p.Q1251E | NM_ | rs15943 | 0.0037 |
| | | | | | | | | 001001671 | | |
| 2 | 0.00 | 0.00 | NaN | SAG | nonsynonymous SNV | c.C374T | p.T125M | NM_000541 | rs137886124 | 0.0024 |
| 3 | 0.00 | 0.00 | NaN | FGFR3 | synonymous SNV | c.C1014T | p.T338T | NM_000142 | rs4647928 | 0.0045 |
| 4 | 0.00 | 0.00 | NaN | VDAC1 | synonymous SNV | c.T111C | p.N37N | NM_003374 | rs142141751 | 0.0028 |
| 5 | 0.00 | 0.00 | NaN | CEL | nonsynonymous SNV | c.G1510C | p.D504H | NM_001807 | rs202171778 | 0.0027 |
| 6 | 0.00 | 0.00 | NaN | SLC14A1 | nonsynonymous SNV | c.G226A | p.V76I | NM_ | rs113029149 | 0.0068 |
| | | | | | | | | 001146036 | | |
| 7 | 0.15 | 0.02 | 1.22 | RSF1 | synonymous SNV | c.G3840A | p.E1280E | NM_016578 | rs11607608 | 0.0052 |
| 8 | 0.17 | 0.02 | 1.40 | DOCK5 | nonsynonymous SNV | c.C1406T | p.T469M | NM_024940 | rs61732769 | 0.0058 |
| 9 | 0.17 | 0.02 | 1.40 | AMN | nonsynonymous SNV | c.A829G | p.T277A | NM_030943 | rs146499374 | 0.0049 |
| 10 | 0.17 | 0.02 | 1.40 | MFSD12 | nonsynonymous SNV | c.G1183A | p.G395S | NM_174983 | rs34878396 | 0.0035 |
| 11 | 0.17 | 0.02 | 1.40 | CEP250 | nonsynonymous SNV | c.G6016A | p.D2006N | NM_007186 | rs61729988 | 0.0054 |
| 12 | 0.20 | 0.02 | 1.63 | HDLBP | nonsynonymous SNV | c.A40T | p.T14S | NM_ | rs116445550 | 0.0033 |
| | | | | | | | | 001243900 | | |
| 13 | 0.20 | 0.02 | 1.63 | FARP2 | nonsynonymous SNV | c.C133T | p.H45Y | NM_ | rs61739735 | 0.0036 |
| | | | | | | | | 001282984 | | |
| 14 | 0.20 | 0.02 | 1.63 | NPY4R | synonymous SNV | c.G516C | p.L172L | NM_005972 | rs140965359 | 0.0024 |
| 15 | 0.20 | 0.02 | 1.63 | RAPSN | nonsynonymous SNV | c.C264A | p.N88K | NM_005055 | rs104894299 | 0.0016 |
| 16 | 0.20 | 0.02 | 1.63 | MRPS23 | synonymous SNV | c.C426T | p.H142H | NM_016070 | rs117066436 | 0.0044 |
| 17 | 0.20 | 0.02 | 1.63 | CEP250 | nonsynonymous SNV | c.G2641A | p.E881K | NM_007186 | rs140439099 | 0.0052 |
| 18 | 0.31 | 0.07 | 1.44 | SLC26A9 | nonsynonymous SNV | c.G514A | p.V172M | NM_052934 | rs146704092 | 0.0064 |

TABLE 2

| | Damaging- | Chromo- | | REF | ALT | LupSuccess- | LupFailure- | | gnomNFE- | |
|---|---|---|---|---|---|---|---|---|---|---|
| # | hits | some | Position | Allele(0) | Allele(1) | Freq | Freq | EndoFreq | freq | p |
| 19 | 0 | chr16 | 2147421 | C | T | 0.0215 | 0.0065 | 0.0072 | 0.0048 | 1.03E−01 |
| 20 | 0 | chr1 | 34112303 | G | A | 0.0153 | 0.0043 | 0.0155 | 0.0072 | 1.33E−01 |
| 21 | 3 | chr1 | 161180482 | G | A | 0.0153 | 0.0043 | 0.0064 | 0.0043 | 1.33E−01 |
| 22 | 1 | chr2 | 219884332 | G | A | 0.0153 | 0.0043 | 0.0087 | 0.0055 | 1.33E−01 |
| 23 | 6 | chr3 | 58145348 | T | C | 0.0153 | 0.0043 | 0.0117 | 0.0087 | 1.33E−01 |
| 24 | 1 | chr4 | 30725272 | C | T | 0.0215 | 0.0043 | 0.0093 | 0.0063 | 3.81E−02 |
| 25 | 2 | chr1 | 3703589 | C | T | 0.0123 | 0.0022 | 0.0070 | 0.0041 | 1.66E−01 |
| 26 | 0 | chr1 | 154936324 | C | T | 0.0123 | 0.0022 | 0.0055 | 0.0030 | 1.66E−01 |
| 27 | 0 | chr1 | 230492931 | G | A | 0.0123 | 0.0022 | 0.0089 | 0.0063 | 1.66E−01 |
| 28 | 1 | chr2 | 186661602 | A | G | 0.0123 | 0.0022 | 0.0064 | 0.0040 | 1.66E−01 |
| 29 | 0 | chr7 | 6026827 | G | C | 0.0123 | 0.0022 | 0.0061 | 0.0037 | 1.66E−01 |
| 30 | 5 | chr7 | 150840440 | A | T | 0.0123 | 0.0022 | 0.0051 | 0.0028 | 1.66E−01 |
| 31 | 0 | chr11 | 128842742 | C | T | 0.0123 | 0.0022 | 0.0032 | 0.0018 | 1.66E−01 |
| 32 | 0 | chr17 | 14204868 | C | T | 0.0123 | 0.0022 | 0.0059 | 0.0027 | 1.66E−01 |
| 33 | 0 | chr21 | 45389040 | C | T | 0.0123 | 0.0022 | 0.0038 | 0.0020 | 1.66E−01 |
| 34 | 0 | chr22 | 21991307 | A | G | 0.0123 | 0.0022 | 0.0042 | 0.0023 | 1.66E−01 |
| 35 | 2 | chr22 | 50435827 | C | T | 0.0123 | 0.0022 | 0.0097 | 0.0069 | 1.66E−01 |
| 36 | 0 | chrX | 19983256 | G | T | 0.0123 | 0.0022 | 0.0091 | 0.0060 | 1.66E−01 |
| 37 | 0 | chrX | 114541176 | G | A | 0.0123 | 0.0022 | 0.0044 | 0.0004 | 1.66E−01 |
| 38 | 1 | chr1 | 34090751 | T | A | 0.0153 | 0.0022 | 0.0078 | 0.0055 | 8.72E−02 |
| 39 | 3 | chr15 | 78461324 | C | T | 0.0153 | 0.0022 | 0.0070 | 0.0040 | 8.72E−02 |
| 40 | 0 | chr11 | 94759863 | G | A | 0.0184 | 0.0022 | 0.0076 | 0.0037 | 2.25E−02 |
| 41 | 0 | chr1 | 226019599 | T | C | 0.0123 | 0.0000 | 0.0038 | 0.0022 | 2.93E−02 |
| 42 | 4 | chr8 | 70536177 | A | G | 0.0123 | 0.0000 | 0.0013 | 0.0003 | 2.93E−02 |
| 43 | 2 | chr9 | 4662929 | G | T | 0.0123 | 0.0000 | 0.0034 | 0.0020 | 2.93E−02 |
| 44 | 2 | chr16 | 20975870 | C | T | 0.0123 | 0.0000 | 0.0034 | 0.0020 | 2.93E−02 |
| 45 | 0 | chr17 | 62517583 | A | G | 0.0123 | 0.0000 | 0.0030 | 0.0017 | 2.93E−02 |
| 46 | 2 | chr17 | 79254530 | C | T | 0.0123 | 0.0000 | 0.0061 | 0.0037 | 2.93E−02 |
| 47 | 0 | chr11 | 5729419 | T | C | 0.0153 | 0.0000 | 0.0102 | 0.0075 | 1.21E−02 |
| 48 | 0 | chr17 | 43923410 | C | T | 0.0184 | 0.0000 | 0.0119 | 0.0071 | 4.95E−03 |
| | | | | | | | | | | gnomad- |
| | | | | | | | | | | population- |
| # | OR | L95 | U95 | gene | type | nuclechange | aachange | transcript | dbsnp | Freq |
| 19 | 3.34 | 0.86 | 13.02 | PKD1 | nonsynonymous SNV | c.G10301A | p.R3434Q | NM_000296 | rs140189010 | 0.0025 |
| 20 | 3.57 | 0.69 | 18.50 | CSMD2 | synonymous SNV | c.C4599T | p.L1533L | NM_052896 | rs10798976 | 0.0321 |
| 21 | 3.57 | 0.69 | 18.50 | NDUFS2 | nonsynonymous SNV | c.G968A | p.R323Q | NM_004550 | rs35086265 | 0.0048 |
| 22 | 3.57 | 0.69 | 18.50 | CCDC108 | synonymous SNV | c.C3369T | p.T1123T | NM_194302 | rs145661891 | 0.0033 |
| 23 | 3.57 | 0.69 | 18.50 | FLNB | nonsynonymous SNV | c.T6956C | p.I2319T | NM_001457 | rs116826041 | 0.0074 |
| 24 | 5.03 | 1.04 | 24.35 | PCDH7 | nonsynonymous SNV | c.C2228T | p.T743I | NM_ | rs36037995 | 0.0051 |
| | | | | | | | | 001173523 | | |
| 25 | 5.70 | 0.63 | 51.25 | LRRC47 | nonsynonymous SNV | c.G901A | p.V301M | NM_020710 | rs138729139 | 0.0024 |
| 26 | 5.70 | 0.63 | 51.25 | SHC1 | synonymous SNV | c.G1710A | p.A570A | NM_ | rs11552298 | 0.0031 |
| | | | | | | | | 001130040 | | |
| 27 | 5.70 | 0.63 | 51.25 | PGBD5 | synonymous SNV | c.C468T | p.D156D | NM_ | rs114204934 | 0.01 |
| | | | | | | | | 001258311 | | |
| 28 | 5.70 | 0.63 | 51.25 | FSIP2 | nonsynonymous SNV | c.A10006G | p.R3336G | NM_173651 | rs142306380 | 0.0021 |
| 29 | 5.70 | 0.63 | 51.25 | PMS2 | synonymous SNV | c.C1569G | p.S523S | NM_000535 | rs141458772 | 0.0058 |
| 30 | 5.70 | 0.63 | 51.25 | AGAP3 | nonsynonymous SNV | c.A2286T | p.E762D | NM_031946 | rs145553128 | 0.0015 |
| 31 | 5.70 | 0.63 | 51.25 | ARHGAP32 | nonsynonymous SNV | c.G3617A | p.R1206H | NM_ | rs139276969 | 0.0011 |
| | | | | | | | | 001142685 | | |
| 32 | 5.70 | 0.63 | 51.25 | HS3ST3B1 | synonymous SNV | c.C33T | p.C11C | NM_006041 | rs201721482 | 0.0014 |
| 33 | 5.70 | 0.63 | 51.25 | AGPAT3 | synonymous SNV | c.C390T | p.P130P | NM_020132 | rs75532875 | 0.004 |
| 34 | 5.70 | 0.63 | 51.25 | CCDC116 | nonsynonymous SNV | c.A1790G | p.D597G | NM_152612 | rs150451119 | 0.0013 |
| 35 | 5.70 | 0.63 | 51.25 | IL17REL | nonsynonymous SNV | c.G896A | p.C299Y | NM_ | rs149914294 | 0.0107 |
| | | | | | | | | 001001694 | | |
| 36 | 5.70 | 0.63 | 51.25 | CXorf23 | nonsynonymous SNV | c.C1180A | p.Q394K | NM_198279 | rs41309713 | 0.0042 |
| 37 | 5.70 | 0.63 | 51.25 | LUZP4 | nonsynonymous SNV | c.G749A | p.R250K | NM_016383 | rs181976775 | 0.0003 |
| 38 | 7.15 | 0.83 | 61.49 | CSMD2 | nonsynonymous SNV | c.A5280T | p.E1760D | NM_052896 | rs35761029 | 0.0162 |
| 39 | 7.15 | 0.83 | 61.49 | IDH3A | nonsynonymous SNV | c.C1078T | p.R360C | NM_005530 | rs116374996 | 0.0023 |
| 40 | 8.61 | 1.03 | 71.83 | KDM4E | nonsynonymous SNV | c.G1142A | p.C381Y | NM_ | rs56043170 | 0.0032 |
| | | | | | | | | 001161630 | | |
| 41 | Inf | NaN | Inf | EPHX1 | synonymous SNV | c.T303C | p.F101F | NM_ | rs2234699 | 0.0015 |
| | | | | | | | | 001136018 | | |
| 42 | Inf | NaN | Inf | SULF1 | nonsynonymous SNV | c.A1595G | p.K532R | NM_ | rs149298828 | 0.0002 |
| | | | | | | | | 001128206 | | |
| 43 | Inf | NaN | Inf | PPAPDC2 | nonsynonymous SNV | c.G554T | p.G185V | NM_203453 | rs149516642 | 0.0011 |
| 44 | Inf | NaN | Inf | DNAH3 | nonsynonymous SNV | c.G9336A | p.M3112I | NM_017539 | rs34622944 | 0.0033 |
| 45 | Inf | NaN | Inf | CEP95 | nonsynonymous SNV | c.A653G | p.H218R | NM_138363 | rs200178960 | 0.001 |
| 46 | Inf | NaN | Inf | SLC38A10 | nonsynonymous SNV | c.G505A | p.V169M | NM_138570 | rs145662017 | 0.0031 |
| 47 | Inf | NaN | Inf | TRIM22 | synonymous SNV | c.T790C | p.L264L | NM_006074 | rs112606816 | 0.005 |
| 48 | Inf | NaN | Inf | SPPL2C | synonymous SNV | c.C1138T | p.L380L | NM_175882 | rs150431364 | 0.0044 |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method comprising:
(a) detecting a presence of at least one genetic variant of Table 1 in a genetic material obtained from a subject, wherein the subject has endometriosis or is at risk of developing endometriosis; and
(b) treating the subject for the endometriosis with a therapeutically effective amount of a treatment that comprises leuprolide acetate, a derivative thereof, a biosimilar thereof, or an interchangeable thereof.

2. A method comprising:
(a) detecting a presence of at least one genetic variant of Table 2 in a genetic material obtained from a subject, wherein the subject has endometriosis or is at risk of developing endometriosis; and
(b) treating the subject for the endometriosis with a therapeutically effective amount of a treatment that does not comprise leuprolide acetate.

3. A method comprising: detecting a presence of at least one genetic variant of Table 1 in a genetic material obtained from a subject, wherein the subject has endometriosis or is at risk of developing endometriosis, wherein the presence of the at least one genetic variant of Table 1 is indicative of a therapeutically effective response to a treatment for treating the endometriosis, and wherein the treatment comprises leuprolide acetate, a derivative thereof, a biosimilar thereof, or an interchangeable thereof.

4. A method comprising: detecting a presence of at least one genetic variant of Table 2 in a genetic material obtained from a subject, wherein the subject has endometriosis or is a risk of developing endometriosis, wherein the presence of the at least one genetic variant of Table 2 is indicative of a therapeutically effective response to a treatment for treating the endometriosis, wherein the treatment does not comprise leuprolide acetate.

5. The method of claim 3, further comprising: treating the subject for the endometriosis.

6. The method of claim 3, wherein the treating comprises prophylactic treating.

7. The method of claim 3, further comprising: altering or updating the treatment based at least in part on the detecting.

8. The method of claim 3, wherein the detecting occurs prior to administering the treatment to the subject.

9. The method of claim 3, further comprising: selecting the treatment from a plurality of treatments.

10. The method of claim 3, further comprising: obtaining the genetic material from the subject.

11. The method of claim 3, further comprising: providing a recommendation to prescribe the treatment to the subject.

12. The method of claim 3, wherein the subject has the endometriosis.

13. The method of claim 3, wherein the subject is at risk of developing the endometriosis.

14. The method of claim 3, wherein the subject suffers from pelvic pain.

15. The method of claim 3, wherein the subject suffers from infertility.

16. The method of claim 3, wherein the genetic material is obtained from a reproductive tissue, a blood sample, or a combination thereof.

17. The method of claim 16, wherein the genetic material is obtained from the reproductive tissue that comprises endometrial tissue, uterine tissue, ovarian tissue, fallopian tissue, cervical tissue, vulvar tissue, or any combination thereof.

18. The method of claim 17, wherein the genetic material is obtained from the reproductive tissue that comprises the endometrial tissue.

19. The method of claim 16, wherein the genetical material is obtained from the blood sample.

20. The method of claim 3, wherein the genetic material comprises cell-free DNA.

21. The method of claim 3, wherein the genetic material comprises RNA.

22. The method of claim 3, wherein the genetic variant comprises at least two genetic variants.

23. The method of claim 3, wherein the genetic variant is of MAP3K15.

24. The method of claim 3, wherein the genetic variant is of C17orf53, MTL5, SYT15, BCO2, ADD1, C14orf79, or any combination thereof.

25. The method of claim 3, wherein the detecting comprises sequencing at least a portion of the genetic material.

26. The method of claim 3, wherein the detecting comprises hybridizing a probe to a portion of the genetic material, wherein the probe is specific for the genetic variant.

27. The method of claim 3, further comprising: measuring a total variant burden in at least a portion of the genetic material.

28. The method of claim 3, further comprising: measuring a mood of the subject.

29. The method of claim 3, further comprising: measuring a hormone receptor level in the genetic material.

30. The method of claim 29, wherein the hormone receptor level is an estrogen receptor level, a progesterone receptor level, or a combination thereof.

31. The method of claim 30, wherein the hormone receptor level is the estrogen receptor level.

32. The method of claim 30, wherein the hormone receptor level is the progesterone receptor level.

33. The method of claim 3, wherein the treatment comprises administration of a gonadotropin releasing hormone (GnRH) or a synthetic analog thereof to the subject.

34. The method of claim 3, wherein the treatment comprises administration of a GnRH receptor agonist, a GnRH receptor antagonist, a progestin, norethindrone, medroxyprogesterone, a biosimilar of any of these, an interchangeable of any of these, a salt of any of these, or any combination thereof.

35. The method of claim 3, wherein the treatment comprises administration of RU-486 (CAS #84371-65-3), ethylnorgestrienone (CAS #16320-04-0), 2,3-isoxazolethisterone (CAS #17230-88-5), elagolix (CAS #834153-87-6), goserelin (CAS #65807-02-5), norethindrone acetate (CAS #38673-38-0), methylhydroxyprogesterone acetate (CAS #71-58-9), a biosimilar of any of these, an interchangeable of any of these, a salt of any of these, or any combination thereof.

36. The method of claim 3, wherein the treatment comprises administration of a pharmaceutical composition in unit dose form.

37. The method of claim 3, wherein the treatment comprises administration of a stem cell.

38. The method of claim 3, wherein the treatment comprises administration of composition comprising: a cannabis, a nonsteroidal anti-inflammatory drug (NSAID), a progestin, a progesterone, or any combination thereof.

39. The method of claim 38, wherein the composition comprises the cannabis, the NSAID, and the progestin.

40. The method of claim 38, wherein the composition comprises the cannabis, the NSAID, and the progesterone.

41. The method of claim 38, wherein the NSAID comprises ibuprofen, naproxen, or a combination thereof.

42. The method of claim 36, wherein the composition further comprises human serum albumin.

43. The method of claim 3, further comprising: comparing a result of the method to a reference.

44. The method of claim 43, wherein the reference comprises a derivative of the reference.

45. The method of claim 43, wherein the reference comprises a result of the method performed on a reference sample.

46. The method of claim 45, wherein the reference sample is of a subject responsive to the treatment.

47. The method of claim 43, wherein the comparing is performed by a computer processor.

48. The method of claim 43, wherein the comparing is performed by a trained algorithm.

49. The method of claim 43, wherein the reference comprises a result obtained from genetic material of a subject diagnosed with endometriosis.

50. The method of claim 43, wherein the reference comprises a result obtained from genetic material of a subject responsive to the treatment.

51. The method of claim 3, further comprising: detecting an epigenetic marker in at least a portion of the genetic material.

52. The method of claim 51, wherein the epigenetic marker comprises a methylated marker, a hydroxymethylated marker, a carboxylated marker, a formylated marker, or any combination thereof.

53. The method of claim 51, wherein the portion comprising the epigenetic marker is RNA or DNA.

54. The method of claim 3, further comprising: reporting a result of the method.

55. The method of claim 54, wherein the result comprises an output of the detecting.

56. The method of claim 54, wherein the reporting comprises electronic reporting.

57. The method of claim 1, further comprising: identifying the subject as a responder to the leuprolide acetate, the derivative thereof, the biosimilar thereof, or the interchangeable thereof.

58. The method of claim 2, further comprising: identifying the subject as a non-responder to the leuprolide acetate.

59. The method of claim 57, wherein the identifying is based in part on: a disease activity score; a presence, an absence, or a recurrence of pelvic pain; a cessation of the treatment; a scoring of dysmenorrhea; a presence of dyspareunia; a failure to conceive; a recurrence of a symptom following a treatment; a surgical intervention; or any combination thereof.

60. The method of claim 59, wherein the identifying is based on the presence, the absence, or the recurrence of pelvic pain.

61. The method of claim 60, wherein the presence, the absence or the recurrence of pelvic pain is reported by the subject on a visual analog scale (VAS).

62. The method of claim 60, wherein the presence, the absence or the recurrence of pelvic pain is reported after the treatment is completed.

63. The method of claim 60, wherein the pelvic pain comprises non-menstrual pelvic pain.

64. The method of claim 59, wherein the identifying is based on the disease activity score.

65. The method of claim 57, wherein the identifying is based at least in part on a medical history of the subject, a hormone receptor level of the subject, a mood of the subject, or any combination thereof.

66. The method of claim 57, wherein the subject is identified as the responder with a sensitivity of at least about 80%.

67. The method of claim 57, wherein the subject is identified as the responder with a specificity of at least about 80%.

68. The method of claim 60, wherein the subject is identified as the non-responder with a sensitivity of at least about 80%.

69. The method of claim 60, wherein the subject is identified as the non-responder with a specificity of at least about 80%.
